(19) United States
(12) Patent Application Publication
(10) Pub. No.: US 2011/0065181 A1
(43) Pub. Date: Mar. 17, 2011

(54) Methods and Apparatus for Segregation of Particles

(75) Inventors: George HVICHIA, Philadelphia, PA (US); David Counts, Royersford, PA (US); Gary Evans, Earlysville, VA (US)

(73) Assignee: Parsortix, Inc., Philadelphia, PA (US)

(21) Appl. No.: 12/910,299

(22) Filed: Oct. 22, 2010

Related U.S. Application Data
(63) Continuation-in-part of application No. PCT/US2009/002421, filed on Apr. 17, 2009.
Application No. PCT/US2009/002421 is a continuation-in-part of application No. PCT/US2010/046350, filed on Aug. 23, 2010.
(60) Provisional application No. 61/125,168, filed on Apr. 23, 2008.
(60) Provisional application No. 61/236,205, filed on Aug. 24, 2009.
(60) Provisional application No. 61/264,918, filed on Nov. 30, 2009.

(51) Int. Cl. C12N 5/073; C12M 1/00; C12M 1/12

(52) U.S. Cl. 435/325; 435/283.1

(57) ABSTRACT

The disclosure relates to an apparatus for segregating particles on the basis of their ability to flow through a stepped passageway. At least some of the particles are accommodated in a passage bounded by a first step, but at least some of the particles are unable to pass through a narrower passage bounded by a second step, resulting in segregation of the particles. The apparatus and methods described herein can be used to segregate particles of a wide variety of types. By way of example, they can be used to segregate fetal-like cells from a maternal blood sample such as maternal arterial blood.

### CROSS-REFERENCE TO RELATED APPLICATIONS

This application is a continuation of co-pending international application PCT/US2009/002421, filed 17 Apr. 2009, which is entitled to priority to U.S. provisional application 61/125,168 (filed 23 Apr. 2008, now abandoned); this application is also a continuation-in-part of co-pending international application PCT/US2010/046350, filed 23 Aug. 2010, which is entitled to priority to U.S. provisional application 61/236,205 (filed 24 Aug. 2009, now abandoned); this application also claims the benefit of the filing date of co-pending U.S. provisional patent application No. 61/264,918, filed 30 Nov. 2009; each of the applications listed in this paragraph is incorporated herein by reference in its entirety.

### BACKGROUND OF THE DISCLOSURE

Among the basic operations necessary for studying or using particles is the ability to segregate different types of particles. For example, innumerable applications in the field of cell biology require the ability to segregate cells of one type from cells of another type. Applications in the field of industrial waste management require the ability to segregate solid particles from industrial waste water or waste gasses. Applications in the field of agriculture and food processing require the ability to separate particulate contaminants from particulate food products such as grains.

For example, blood drawn from the umbilicus shortly after delivery (“cord blood”) is a rich source of stem cells, such as embryonic stem cells and hematopoietic stem cells. Hematopoietic stem cells are useful for treating blood disorders. Methods of storing cord blood samples are known. These methods have the drawback that a relatively large volume (e.g., 100 to 250 milliliters) of blood must be stored in order to preserve a sufficient number of stem cells for use in future medical procedures. The large volume of cord blood that is stored increases the cost and decreases the convenience of the procedure. The stored volume could be decreased significantly (e.g., to 0.1 to 1 milliliter) if stem cells could be readily separated from cord blood prior to storage. However, present methods of separating stem cells from cord blood are expensive, cumbersome, and sometimes ineffective. There is a need for an efficient and cost-effective method of segregating stem cells from cord blood.

Further by way of example, cells of apparently fetal origin (i.e., fetal-like cells) can be found in the blood of pregnant women and in the blood of women who have previously been pregnant. These cells can have male DNA when the mother has given birth to or is pregnant with a male child, and therefore the DNA appears to originate from the fetus. These cells are rare in the maternal bloodstream; there may be only 10 to 12 cells per milliliter of maternal blood. Among fetal-like cells observed in maternal blood, fetal trophoblasts can degrade relatively quickly after the woman gives birth. Other kinds of fetal-like cells have been reported to endure in the blood of women for years or decades following pregnancy albeit in small numbers. The rarity and apparently short duration of some fetal-like cells can make them difficult to capture. Consequently, little is known about the cells. A need exists for a way to quickly, economically, and effectively segregate fetal-like cells from maternal blood. A need also exists for a way to segregate fetal trophoblasts from other fetal-like cells in maternal blood.

Mechanical devices intended for manipulation of biological cells and other small particles and having structural elements with dimensions ranging from tens of micrometers (the dimensions of biological cells) to nanometers (the dimensions of some biological macromolecules) have been described. For example, U.S. Pat. No. 5,928,880, U.S. Pat. No. 5,866,345, U.S. Pat. No. 5,744,366, U.S. Pat. No. 5,486,335, and U.S. Pat. No. 5,427,946 describe devices for handling cells and biological molecules. PCT Application Publication number WO 03/008931 describes a microstructure for particle and cell separation, identification, sorting, and manipulation.

Passage of blood through a space, defined in one dimension in microns, presents challenges. Tidal pressure forces which tend to disrupt cellular integrity and potential clogging of the passage space due to “packing” of cells must be taken into account. This is also complicated by the tendency of blood to clot (in a cascading manner) if cellular integrity is compromised. Furthermore, it is known that large particles (cells, agglomerated cells, extracellular materials, and poorly characterized “debris” in biological samples can clog the fluid passages of prior devices, inhibiting their efficiency and operation.

The subject matter disclosed herein can be used to segregate and manipulate biological cells, organelles, and other particles from mixed populations of particles or cells.

### SUMMARY OF THE DISCLOSURE

The present disclosure relates to an apparatus for segregating particles such as cells. The apparatus includes a body, a cover, and a separation element. The body and cover define a void. The separation element is contained within the void. The void has a fluid inlet region and a fluid outlet region. The separation element has a shape that defines a stepped passageway that fluidly connects the inlet and outlet regions in the void. The separation element includes a first step and a second step, each of which extends into the stepped passageway. The passage bounded by the second step is narrower than the passage bounded by the first step. When a fluid including particles is present at the inlet region, fluid can flow from the inlet region, through the first passage, through the second passage, and into the outlet region. Particles suspended in the fluid can transit the first and second passages if the size of the particles does not exceed the narrow dimension of each passage, or if the particles are sufficiently deformable that, in a deformed shape, they can squeeze through each passage. Particles can be segregated by selecting a narrow dimension for the second passage that permits only some of the particles to pass therethrough. The narrow dimension of the first passage can be selected such that particles in the fluid can pass through the first passage individually, but two particles cannot pass through the first passage simultaneously if they are stacked across the narrow dimension of the first passage.

The apparatus can include a fluid inlet port for facilitating fluid flow from outside the apparatus into the inlet region, a fluid outlet port for facilitating fluid from the outlet region to the outside of the apparatus, or both. A fluid displacement device (e.g., a pump or a gravity-fed fluid reservoir can be fluidly connected with one or both of the inlet and outlet ports to facilitate fluid flow through the stepped passageway. Such flow can be in the direction from the inlet region toward the outlet region, for the purpose of segregating particles. Fluid flow can be in the direction from the outlet region toward the inlet region, for example to flush out particles that were unable to traverse the second passage during inlet region-to-outlet region fluid flow.

The steps of the separation element define passages within the stepped passageway, and there can be two or more such steps. The steps can be formed from planar regions that meet at a right angle (forming classical right-angled steps), or the riser portion (i.e. the transitional face) of the step can be inclined, such that a first planar step region can be connected to a second planar step region by a sloped flat surface or by a curved surface. The planar step regions can be substantially parallel to a portion of the cover, a portion of the body, or both, and should have a length (in the direction of bulk fluid flow) equal to a multiple (e.g., 2, 4, 10, or 1000) of the narrow dimension of the passage it bounds. The width of the planar region (in the direction perpendicular to bulk fluid flow) should be equal to a multiple (e.g., 10, 1000, of 10000) of the narrow dimension of the passage it bounds.

The apparatus can have one or more supports within the void for maintaining the dimensions of the stepped passageway during assembly and operation of the device. The support can completely span the distance between the separation element the body or the cover or it can span only a portion of that distance, to provide room for deformation of an element (e.g., upon assembly and clamping of the apparatus).

The present disclosure includes a method of segregating particles. The method includes introducing particles at the inlet region of the apparatus, permitting them to move (i.e., by endogenous cell motility or under the influence of induced fluid flow) through a stepped passageway to an outlet region. At least some of the particles are prevented from entering the outlet region by a step in the passageway, resulting in segregation of the particles. Particles able to traverse all steps in the stepped passageway can be collected from the outlet region. Particles unable to traverse at least one step in the stepped passageway can be collected from a portion of the passageway upstream from the step that inhibits their movement through the passageway. For example, trapped particles can be recovered by inserting a device (e.g., a catheter) into the stepped passageway, by reversing fluid flow and flushing the trapped cells out of the passageway by way of the inlet region, or by disassembling the device and recovering the trapped particles directly. If the trapped particles are cells, they can be lysed within the stepped passageway and the lysis products collected by flow in either direction.

### BRIEF DESCRIPTION OF THE SEVERAL VIEWS OF THE DRAWINGS

The foregoing summary, as well as the following detailed description of the invention, will be better understood when read in conjunction with the appended drawings. These drawings are included for the purpose of illustrating the disclosure. The disclosure is not limited to the precise arrangements and instrumentalities shown.

FIG. 1 consists of FIGS. 1A and 1B. FIG. 1A is an elevated view of a portion of the apparatus in one embodiment. FIG. 1B is a vertical section of the portion of the apparatus shown in FIG. 1A, taken along plane 1B, showing a body 10 which defines a void 11. A cover 12 is disposed across the void 11 forming a fluid-tight seal with the body 10. A separation element 14 having a first step 61 and a second step 62 is disposed within the void 11 between an inlet port 16 and an outlet port 18. The first step 61 has a broad surface 31 and a transitional face 41. The second step 62 has a broad surface 32 and a transitional face 42.

FIG. 2 consists of FIGS. 2A, 2B, and 2C. FIG. 2A is an elevated view of a portion of the apparatus in an embodiment having inner support structures 20. FIG. 2B is a vertical section of the portion of the apparatus shown in the FIG. 2A, taken along plane 2B. FIG. 2C is a vertical section of a portion of the apparatus shown in FIG. 2A, taken along plane 2C.

FIG. 3 consists of FIGS. 3A and 3B and illustrates a configuration of the apparatus described herein wherein the geometry of the first and second passages can be selected to achieve substantially constant linear flow velocity throughout the first and second passages. FIG. 3A is an elevated view of a series of passages wherein the width of each passage increases in the direction from the inlet region to the outlet region. FIG. 3B is a vertical section of the series of passages shown in FIG. 3A taken along plane 3B, wherein the height of each passage decreases in the direction from the inlet region to the outlet region.

FIG. 4 is a perspective view of a portion of a separation element showing the length “l”, height “h”, and width “w” of a step, and indicating the direction of bulk fluid flow “BFF” past the step.

FIG. 5 is a color image showing an elevated view of the cover 12 of an assembled apparatus, showing the light pattern in an appropriately assembled apparatus, as described herein in Example 2.

FIG. 6 is a diagram that illustrates the relative arrangements of the cover 12, base 10, and first, second, third, fourth, fifth, sixth, seventh and eighth steps (61-68) of the separation element 14 of an apparatus used in experiments described herein in Examples 3 and 4. The direction of fluid flow is shown as ‘D.’

FIG. 7 is a map showing the approximate locations within the separation region of the experiments described herein in Example 4 at which fetal-like cells were found. The portion of the Relative Vertical Position designated “Outlet Area” corresponds approximately to the portion of the cassette at which steps having cover-to-step distances of 4.2 and 4.4 micrometers were located, and the portion of the Relative Vertical Position designated “Inlet Area” corresponds approximately to the portion of the cassette at which steps having cover-to-step distances of 5.2 and 5.4 micrometers were located.

FIG. 8 consists of FIGS. 8A and 8B. FIG. 8A is an elevated view of one embodiment of a portion of membrane 81. FIG. 8B is a magnified view of the portion of the vertical section of the membrane 81 from FIG. 8A, taken along plane 8B. In this particular embodiment, membrane 81 is porous and coated on one side, therefore FIG. 8B shows pores 82 extending through membrane 81 and coating 83. In embodiment shown in FIG. 8, the coating 83 is applied to one face of membrane 81, but not the other face, does not extend into pores 82, and does not fill pores 82.

FIG. 9 is a vertical section, taken along plane 9-9 in FIG. 2A, of a device of the type shown FIG. 2A and including a membrane 81 interposed between the body 10 and the cover 12. Inner supports 20 aid in even, leveled displacement of membrane 81 within the device. Inner supports 20 also help to define the void 11, and provide control over the size of the void 11.

### DETAILED DESCRIPTION

The disclosure relates to an apparatus for segregating particles on the basis of their ability to traverse a passage. Particles (e.g., particles suspended in a liquid or gaseous fluid or particles in a vacuum) are moved through a stepped passageway defined by a separation element in the apparatus. The stepped passageway contains at least two passages that are fluidly connected in series, each passage having a narrow dimension. Most or all particles in the fluid are able to move into the first passage, but only some of the particles are able to move through the second passage. The net result is that some particles can move through the entire stepped passageway, while other particles are retained within the apparatus, such as within the first passage. Segregation of particles is thus achieved. Movement of particles can be motivated by fluid flow, gravity, vibration, or any combination of these, for example.

A membrane or other semi-permeable or penetratable barrier can be used in combination with the apparatus to segregate particles able to cross the barrier from particles unable, less able, or less quickly able to cross the barrier. In this embodiment, the apparatus can be used to segregate particles both on the basis of their ability to traverse the passage and their ability to traverse the membrane. One or more portions of the apparatus (including the membrane) can be coated with a reagent that specifically binds with particles of interest to enhance recovery, segregation, or both, of desired particles.

### DEFINITIONS

As used herein, each of the following terms has the meaning associated with it in this section.

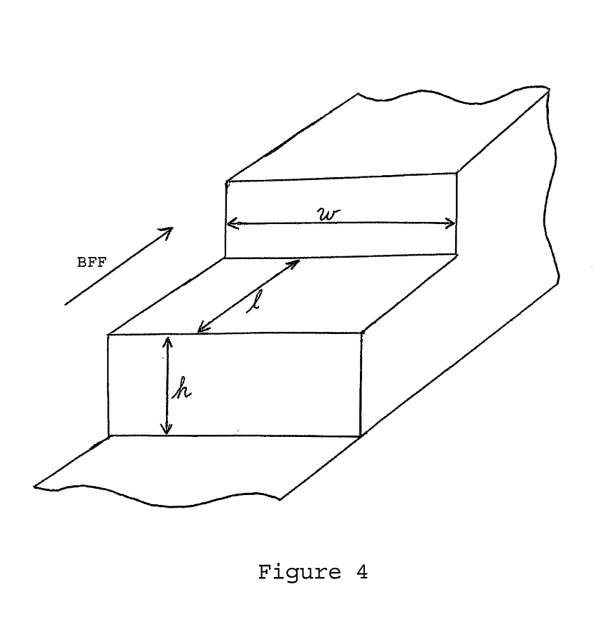

As illustrated for rectangular steps in FIG. 4, the “length” of a step (or of the passage bounded by the step; “l” in FIG. 4) refers to the distance that the step extends in the direction of bulk fluid flow through the passage corresponding to the step.

As illustrated for rectangular steps in FIG. 4, the “height” of a step (“h” in FIG. 4) refers to the distance that the step extends in the direction away from the separation element beyond the preceding (i.e., upstream) step surface.

As illustrated for rectangular steps in FIG. 4, the “width” of a step (or of the passage bounded by the step; “w” in FIG. 4) refers to the distance that the step extends in the direction that is perpendicular to bulk fluid flow over the step.

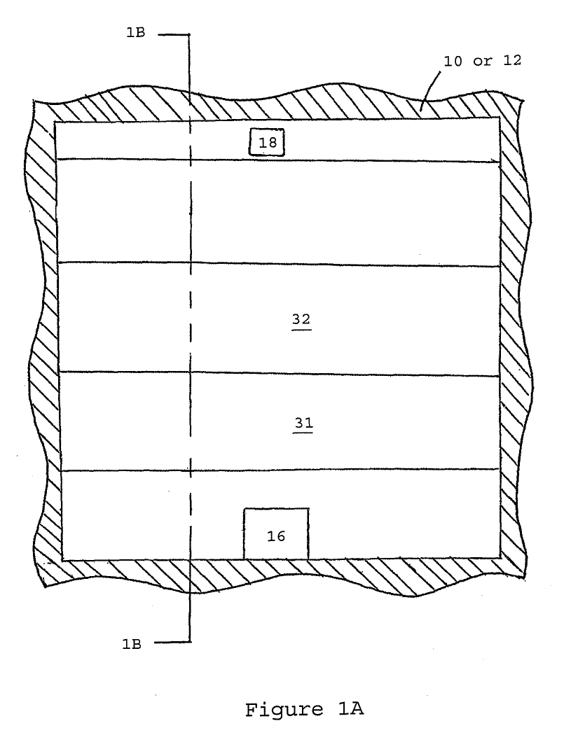

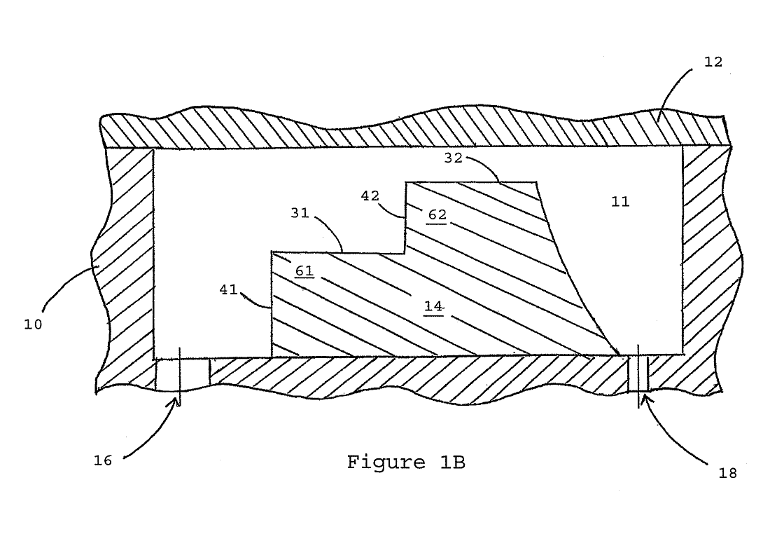

The “narrow dimension” of a passage refers to the distance between the broad portion of a step of the separation element and the opposed, generally parallel, face of the apparatus (e.g., the face of the cover or body that faces the void). For example, for a passage having a rectangular cross-section in a plane taken perpendicular to the direction of bulk fluid flow through the passage, the narrow dimension of the passage is the length of a line in that plane extending between and at right angles to each of the flat surface of the step and the flat surface of the opposed face of the apparatus. Further by way of example, the “narrow dimension” of each of passages 51 and 52 in FIG. 1B is the minimum distance between each of the step surfaces 31 and 32 and the nearest surface of cover 12.

The “flow area” of a passage is a cross-section of the passage taken in a plane perpendicular to the direction of fluid flow in the passage.

### DETAILED DESCRIPTION

The disclosure relates to an apparatus for segregating particles on the basis of their ability to flow through at least two passages, the second (downstream) passage 52 being narrower than the first (upstream) passage 51. The apparatus includes a separation element 14 disposed in a void 11 formed by a body 10 and cover 12. Within the void 11, the separation element 14 separates an inlet region 15 of the void from an outlet region 17 of the void. The inlet and outlet regions are in fluid communication by way of a stepped passageway defined by the separation element 14 and one or both of the body 10 and cover 12. Steps formed in the separation element define the first and second passages. The apparatus optionally has an inlet port 16 that fluidly communicates with the inlet region 15 of the void 11 and an outlet port 18 that fluidly communicates with the outlet region 17 of the void 11, to facilitate provision and withdrawal of fluid to the inlet and outlet regions.

In one embodiment, the apparatus includes a membrane or other barrier 81 that is in fluid communication with the void 11 and that is selectively permeable to particles of a desired type, relative to particles of another type. Alternatively, the membrane or other barrier 81 can have attached thereto a reagent that selectively binds with particles of a desired type, relative to particles of another type. In an apparatus including both the separation element 14 and a membrane or other barrier 81, the separation element and membrane or other barrier 81 can be selected to enhance segregation of the same particle type (i.e., the two elements enhancing segregation of the desired particles) or different particle types (i.e., the two elements promoting segregation of multiple particle types from mixtures of particles, including from one another).

In operation, particles in the inlet region 15 pass into the first passage 51 and, if they are able, into the second passage 52. Particles in the second passage 52 pass to the outlet region 17. Cells that are not able to pass into or along the second passage 52 do not reach the outlet region 17. In this way, particles able to reach the outlet region 17 are segregated from particles that are not able to reach the outlet region 17. The two populations of particles can be separately recovered from the apparatus. For example, particles at the outlet region 17 can be recovered in a stream of liquid withdrawn from the outlet region 17 (e.g., by way of an outlet port or by way of a catheter inserted into the outlet region 17. Particles unable to pass through the second passage 52 to the outlet region 17 can be recovered by flushing them, in the reverse direction, through the first passage 51 and into the inlet region 15. Such particles can be withdrawn from the inlet region 15. Alternatively, particles unable to pass through the second passage 52 to the outlet region 17 can be left in the apparatus or recovered by disassembling the apparatus. Particles unable to enter either the first passage 51 or the second passage 52 can be recovered from the inlet region 15.

The apparatus described herein can be used in a wide variety of applications. In addition to segregating particles from a mixed population of particles, the device can be used in applications in which one or more of the segregated particle populations are identified or further manipulated, for example. The construction and operation of the apparatus resist clogging by the particles being segregated, relative to devices previously used for particle separation. Advantageously, the particles segregated using the apparatus described herein can be suspended in a liquid or gaseous fluid, or in no fluid at all (e.g., in a vacuum). Furthermore, any fluid in which particles are suspended can either be flowed through the apparatus or remain static. That is, particles can be segregated regardless of whether any fluid in which they are suspended is caused to move through the spaces of the apparatus. Thus, for example, particles in a mixture of dry particles can be segregated by providing the mixture to the inlet region and vibrating or shaking the device (oriented such that gravity will tend to draw the particles through the separation region). Such use can be beneficial in situations in which suspension of particles in a fluid is considered undesirable or unnecessary (e.g., when separating plant seeds from other particulate matter such as seeds of other plants).

Parts and portions of the apparatus are now discussed separately in greater detail.

The Body and Cover

The apparatus has a body 10 and a cover 12 defining a void 11 therebetween. A portion of the void 11, defined in part by the separation element 14, is a stepped passageway. The stepped passageway is also defined by a surface of the body 10, a surface of the cover 12, or by a combination of these, that is opposed to the stepped surface(s) 31 and 32 of the separation element 14. (i.e., in an orientation such that the stepped passageway-defining surface(s) of the body 10 and/or cover 12 contact the stepped passageway-defining surface(s) of the separation element 14 in such a way that the surfaces form an extended lumen (i.e., the stepped passageway) between the surfaces. In order to simplify construction of the apparatus, most or all of the stepped passageway-defining surfaces can be formed or machined into a separation element 14 that is an integral part formed in a recess of the cover 12 or the body 10, the recessed portion being surrounded by a flat surface, so that the opposed surface of the body 10 or the cover 12 need only be another flat surface in order to form the stepped passageway upon contact between the flat surfaces of the body 10 and cover 12.

The separation element 14 is preferably integral with (formed or machined as a part of) one of the body 10 and the cover 12. In this embodiment, the operative portion of the apparatus consists of essentially two pieces—either a cover 12 and a body 10 having a separation element 14 as a part thereof, or a body 10 and a cover 12 having a separation element 14 as a part thereof. It is not important which of the body 10 and cover 12 bears the separation element 14, because the body 10 and cover 12 form the walls of and define the void 11 in which the separation element 14 is disposed. Preferably, a portion of the part not bearing the separation element 14 is simply a flat surface that mates with flat edges of the part bearing the separation element 14 and having the void 11 therein, so that upon assembly of the two parts, the void 11 is sealed by mating of the flat surfaces and the separation element 14 is disposed within the thus-sealed void 11. In this embodiment, one of the parts has both the void 11 and the separation element 14 formed or machined therein or, alternatively, has the void 11 formed or machined therein and has the separation element 14 placed, assembled, formed, or adhered within the void 11.

The shapes of the body 10 and cover 12 are not critical, except for the portion(s) of the body 10 and/or cover 12 that define the stepped passageway in the void 11 and the portion(s) of the body 10 and cover 12 that mate to seal the void 11. The requirements of the portion(s) of the body 10 and/or cover 12 that define the stepped passageway are discussed in the section of this disclosure pertaining to the stepped passageway. The portion(s) of the body 10 and cover 12 that mate to seal the void 11 do not have any particular shape or location requirements, other than that they should seal the void 11 when the apparatus is assembled, with allowances for any orifices (e.g., inlet or outlet ports) that are bounded by both the body 10 and the cover 12. Sealing can be achieved by direct contact between the relevant portions of the body 10 and cover 12. Alternatively or in addition, sealants such as adhesives, greases, gaskets, waxes, and the like can be applied on the sealing surfaces of the body 10 and cover 12. The seal should be able to withstand the anticipated internal pressure generated within the apparatus during its operation. For example, in many embodiments, an internal fluid pressure greater than 25 pounds per square inch of gauge pressure (psig) would be unusual, and a seal capable of preventing fluid leaks at this pressure should suffice for such embodiments. More typical operation pressures in embodiments in which biological cells are separated using the apparatus are anticipated to be within the range >0-15 psig. In some embodiments, the apparatus can be operated by application of negative (i.e., vacuum) pressure to the outlet region, in which embodiments the seal should prevent the passage of air or liquid from outside the device into the void (other than, of course, by way of the inlet region).

The size and shape of the remaining portions of the body 10 and cover 12 are not critical and can be selected to facilitate, for example, manufacturing, handling, or operation of the apparatus. By way of example, for an apparatus having a substantially flat cover 12 (e.g., like a cover slip for a microscope slide), the body 10 can have the void 11 and separation element 14 formed or machined therein, and portions of the body 10 outside the void 11 can be formed or machined to adapt the body 10 for securing it in a frame or holder of fixed geometry. Thus, for example, the body 10 can have flanges, handles, threaded holes, smooth bores, impressions or indentations for holding a clamp, or other features formed, applied, or machined therein or thereon, and such features can facilitate reproducible orientation of the body 10 in a device for operating the apparatus or reproducible orientation of the body 10 in a device for machining one or both of the void 11 and the separation element 14 in the body 10.

The body 10, the cover 12, or both can define a port through which fluid can be introduced into or withdrawn from the void 11. For example, the body 10 can define an inlet port 16 that fluidly communicates with the inlet region 15. Fluid introduced into the inlet port 16 can flow into the inlet region 15, displacing fluid already there (because the void is sealed) into the stepped passageway, and thence into the first passage 51 and the second passage 52 and into the outlet region 17. Particles suspended in fluid in one of these regions and passages can be carried into a downstream region or passage, provided the particle can flow through the present and intervening passages and regions. Similarly, withdrawal of fluid from the outlet region 17 by way of an outlet port 18 formed in the body 10 can induce fluid flow from passages in fluid communication with the outlet region 17 and from passages and regions in fluid communication therewith.

Ports can be simple holes which extend through the cover or body, or they can have fixtures (burrs, rings, hubs, or other fittings) associated with them for facilitating connection of a fluid flow device to the port. The body 10, cover 12, or both can define an inlet port 16 in the inlet region 15 of the void 11, an outlet port 18 in the outlet region 17 of the void 11, or both an inlet port 16 and an outlet port 18. Fluid can be introduced into the inlet region 15 through the inlet port 16. Fluid can be withdrawn from the outlet region 17 through the outlet port 18. Continuous introduction of fluid into the inlet region 15 and simultaneous withdrawal or emission of fluid from the outlet region 17 can create a continuous flow of fluid through the apparatus. Similarly, continuous withdrawal of fluid from the outlet region 17 and simultaneous influx or introduction of fluid into the inlet region 15 can create continuous flow.

The Void

The body 10 and the cover 12 form a void 11 when they are assembled. The void 11 has an inlet region 15, an outlet region 17, and a separation region interposed between the inlet region 15 and the outlet region 17. A separation element 14 is disposed within the separation region and, together with the body 10, the cover 12, or both, defines a stepped passageway. The stepped passageway includes at least a first passage 51 and a second passage 52, that are fluidly connected in series and that are defined by steps in the separation element 14. The stepped passageway can include any number of additional steps, each of which can define an additional passage in the void.

During operation of the device, at least the inlet region 15, the outlet region 17, and the stepped passageway of the void 11 are filled with a fluid. Preferably, the entire void 11 is filled with fluid during operation. In one embodiment, the only fluid path that connects the inlet region 15 and the outlet region 17 is the stepped passageway. Particles present in the inlet region 15 can enter and pass through the first passage 51 of the stepped passageway unless they are excluded by the size (i.e., the narrow dimension) or shape of the first passage 51. Particles present in the first passage 51 can enter the second passage 52 unless they are excluded by the size (i.e., the narrow dimension) or shape of the second passage 52, or unless their movement through the first passage 51 is inhibited by the size (i.e., the narrow dimension) or shape of the first passage 51. Particles present in the second passage 52 can enter the outlet region 17 unless their movement through the second passage 52 is inhibited by the size (i.e., the narrow dimension) or shape of the second passage 52. Movement of particles within the apparatus can be induced by fluid flow through the apparatus, by intrinsic motility of the cells, or a combination of the two. Over time, particles unable to enter the first passage 51 will be segregated in the inlet region 15; particles able to enter the first passage 51 but unable to enter the second passage 52 (or to freely move though the first passage 51) will be segregated in the first passage 51; particles able to enter the second passage 52 but unable to freely move therethrough will be segregated in the second passage 52; and particles able to move through both the first passage 51 and the second passage 52 will be segregated in the outlet region 17 (or in fluid withdrawn or emitted from the outlet region 17).

Particles segregated in this manner can be recovered (using any of a variety of known methods, including some described herein) from their respective locations. By way of example, a catheter can be inserted into a region or passageway (e.g., the inlet region 15 or the first passage 51) of the apparatus, and particles present therein can be withdrawn by inducing suction in lumen of the catheter. Further by way of example, backflushing (i.e., fluid flow from the outlet region 17 in the direction of the inlet region 15) can be used to collect particles present in one or more of the inlet region 15, the first passage 51, and the second passage 52 in fluid collected, withdrawn, or emitted at the inlet region 15. Still further by way of example, particles present at the inlet region 15 can be collected by a transverse (relative to bulk fluid flow from the inlet region 15 to the outlet region 17 by way of the stepped passageway) fluid flow across the inlet region 15, using ports provided for this purpose in fluid communication with the inlet region 15.

The Separation Element

Situated in the void 11 defined by the body 10 and the cover 12 and between the inlet region 15 and the outlet region 17 of the void 11, the separation element 14 is a part of the apparatus that has a surface that defines part of the stepped passageway. One or both of the body 10 and the cover 12 define the remaining boundaries of the stepped passageway, which fluidly connects the inlet region 15 and the outlet region 17. The separation element 14 has a shape that includes at least two steps, the steps forming at least one of the boundaries of each of the first passage 51 and the second passage 52. One or both of the body 10 and the cover 12 define the remaining boundaries of the first passage 51 and the second passage 52.

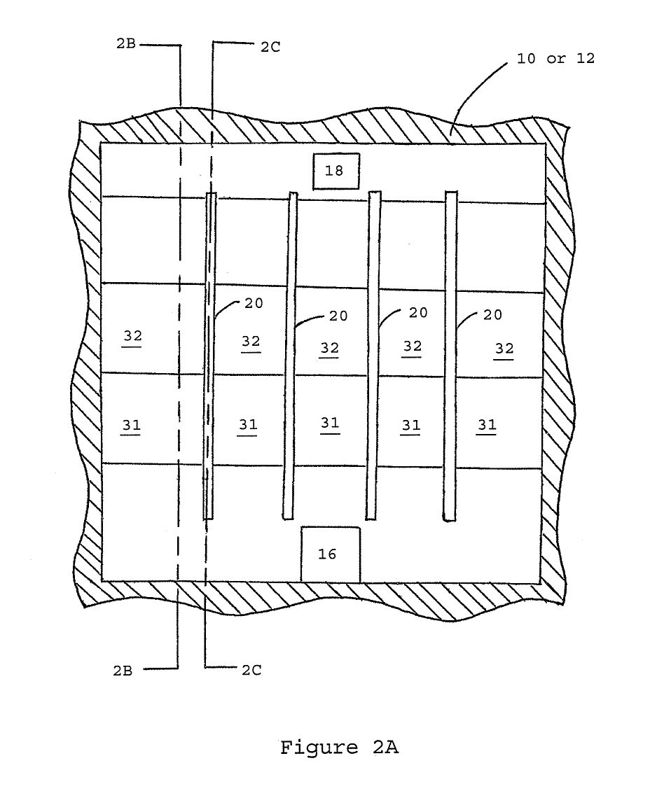

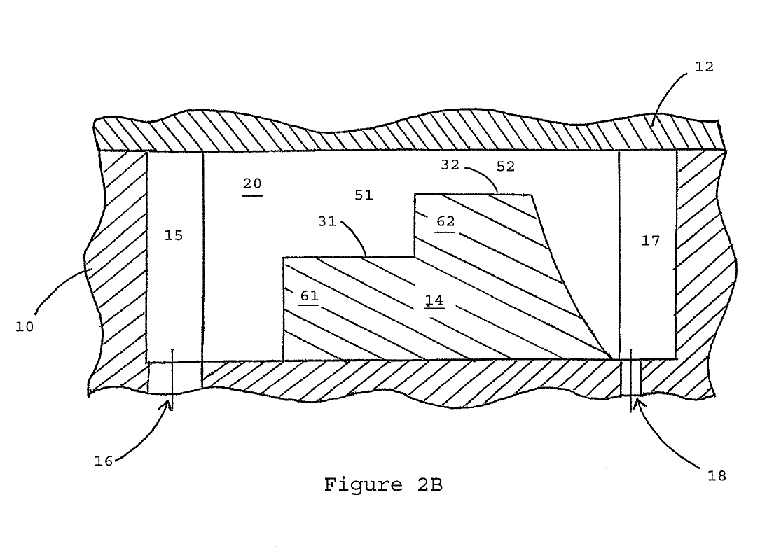

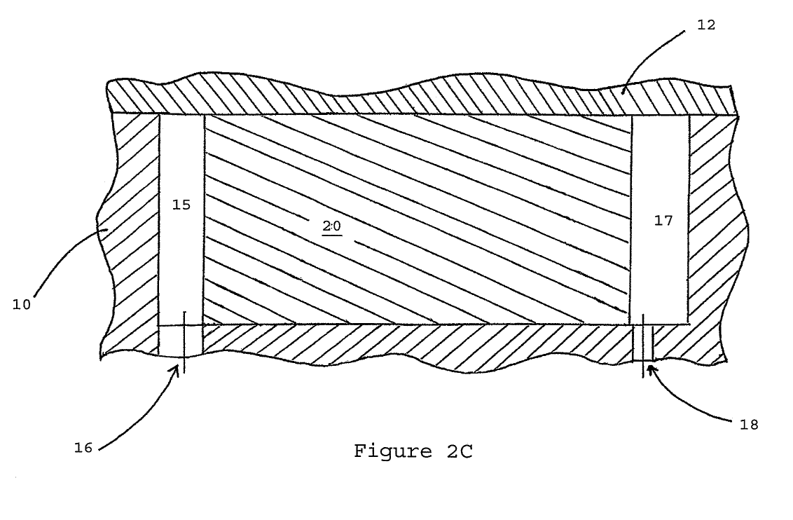

The stepped passageway is the orifice through which particles move, fluid flows, or both, during operation of the apparatus. The separation element 14 has a stepped structure, which defines the stepped shape of at least one side of the stepped passageway. The separation element 14 has at least two steps, the first step 61 and the second step 62. The first step 61 defines a boundary of the first passage 51 in the stepped passageway. The second step 62 defines a boundary of the second passage 52, the second passage 52 having a smaller narrow dimension (see, e.g., FIG. 2B) than the first passage 51. The first and second passages are fluidly connected in series, the second passage 52 being downstream from the first passage 51 during normal operation of the apparatus. Fluid must flow through each of the first and second passages in the stepped passageway in order to travel from the inlet region 15 to the outlet region 17 when the apparatus is assembled.

The separation element 14 is associated with at least one of the body 10 and the cover 12. The separation element 14 can be attached to the surface of the body 10 or the cover 12. The separation element 14 can instead be integral with one of the body 10 or the cover 12, such that when the body 10 and the cover 12 are assembled, the stepped surface(s) of the separation element 14 are brought into opposition with the surface(s) of the body 10 or the cover (12) that form the boundaries of the stepped passageway. Alternatively, the separation element 14 can be a part separate from the cover 12 or the body 10. If the body 10, the cover 12, and the separation element 14 are separate parts, then the parts are preferably dimensioned and shaped such that the separation element 14 is held in place by compression between the cover 12 and the body 10 when the apparatus is assembled.

Fluid pressures within the apparatus (e.g., within the second passage 52) are exerted on all surfaces contacted by the fluid, and such fluid pressures can induce bending or bulging in deformable materials. Furthermore, external pressure applied to parts of the apparatus in order to secure it in its assembled state (e.g., one or more clamps which urge the cover 12 against portions of the body 10) can also induce flexation or bulging in flexible materials that form one or more parts of the apparatus. Because the second passage 52 defined by the separation element 14 and at least one of the body 10 and the cover 12 is the primary mechanism by which particles are segregated by the apparatus in operation, it is preferable that the narrow dimension of the second passage 52 be carefully maintained relatively constant across the width of the second step 62.

By way of example, the second passage 52 has boundaries defined by the second step 62 of the separation element 14 and by one or both of the body 10 and the cover 12. Clamping the body 10 and the cover 12 together can exert external force on a part which forms a boundary of the second passage 52, thereby tending to induce flexation of the part and narrowing of the narrow dimension of the second passage 52. Such flexation and narrowing can be reduced or eliminated by including one or more supports 20 within the lumen of the second passage 52. A support 20 can be, for example, a rod-shaped extension extending from the surface of the separation element 14 that defines the boundary of the second passage 52 in the direction of the opposed surface of the body 10 or the cover 12. Alternatively, an extension having a rectangular cross-section can extend away from the surface of the body 10 or the cover 12 that defines a boundary of the second passage 52 in the direction of the opposed surface of the separation element 14 can form a support 20. More than one support 20 can be arranged in parallel or in series to form one or more solid or segmented walls, and such supports can define multiple flow paths within the void, the multiple flow paths merging at one or both of their ends. As a third alternative, a support 20 can be a discrete part disposed in the lumen of the second passage 52 and substantially or fully spanning the narrow dimension between the opposed surfaces of the separation element 14 and the body 10 or cover 12. Impingement of the support 20 upon the surface of the separation element 14 that defines the second passage 52, upon the surface of the body 10 or cover 12 that defines the second passage 52, or upon both surfaces, limits or halts flexation of the surfaces, maintaining the narrow dimension to a value substantially equal to or greater than the thickness of the support 20 (e.g., to prevent the cover 12 from depressing completely against the broad surface 32 of the second step 62 and reducing the narrow dimension of the second passage 52 below the desired value).

The supports 20 brace the parts of the apparatus in their appropriate conformation, increasing the dimensional stability of the apparatus. By increasing dimensional stability, the supports 20 can enhance the operability of the apparatus under various operating conditions (e.g., with varying clamping pressures or with varying fluid pressures) and extend the life of the apparatus. Supports 20 can also enhance the particle segregating accuracy of the apparatus by preventing the body 10 or cover 12 from deforming and altering the narrow dimensions of one or more of the first and second passages of the stepped passageway. Supports 20 can also be disposed in the void 11 outside of the first and second passages, and span the height of the void. Such supports 20 can maintain the patency of the void 11 outside the first and second passages. Where a support 20 is not integral with a surface impinged by the support 20, the support 20 can be not attached to the surface, adhered to the surface (e.g., using an adhesive interposed between and binding both the surface and a portion of the support), or fused with the surface.

Supports 20 can separate an otherwise unitary fluid flow path into two or more fluid flow paths within the void 11 (see, e.g., supports 20 in FIG. 2A). In an embodiment depicted in FIG. 2, the apparatus consists of a flat cover 12, a body 10 having a flat surface that mates with the cover 12 and defining a void 11 having an inlet region 15 and an outlet region 17, and a separation element 14 that includes a first step 61 and a second step 62 and is integral with four supports 20. When the separation element 14 is disposed in the void 11 between the inlet region 15 and the outlet region 17, the height of the supports 20 is equal to the depth of the void 11, such that the upper surfaces of the supports 20 are substantially co-planar with the flat surface of the body 10 (as depicted in FIGS. 2B and 2C). When the cover 12 is assembled against the flat surface of the body 10, the top surfaces of the supports 20 contact the surface of the cover 12 that defines the void 11, thereby preventing clamping pressure (applied to the cover 12 to hold it flush against the flat surface of the body 10) from deforming the cover 12. The bracing provided to the cover 12 by the supports 20 serves to maintain the narrow dimension of the second passage 52 and the narrow dimension of the first passage 51, even when clamping pressure that would otherwise deflect the cover 12 inwardly toward the void is applied to the cover 12. If the cover 12 is fused with or adhered to one or more of supports 20, then the apparatus depicted in FIG. 2 can also resist expansion of the narrow dimension of the first passage 51 and the second passage 52 that might otherwise result from outward (i.e., away from the void 11) flexation of the cover 12 induced by fluid pressure within the apparatus.

The shape, contour, size, and orientation of the supports 20 are not critical. Supports 20 can have rectangular, rhomboid, circular, elliptical, or wing-shaped cross-sections, for example. In addition to forming walls that direct fluid flow (as do the supports 20 depicted in FIG. 2), supports 20 can induce turbulence in fluid flow paths and induce mixing and or displacement of particles immediately downstream from such supports. By way of example, supports having rounded cross-sections and placed near the leading (i.e., upstream-most) edge of the second passage 52 can induce turbulent flow at the leading edge of the second passage 52, jostling particles that might otherwise occlude the second passage 52 and thereby enhancing fluid flow through the second passage 52.

The separation element 14 can define fluid flow paths other than the stepped passageway discussed herein. Such fluid flow paths can, for example, extend between the inlet region 15 and the stepped passageway or between the stepped passageway and the outlet region 17. Further by way of example, the first passage 51 defined by the first step 61 of the separation element 14 can be connected with the second passage 52 defined by the second step 62 of the separation element 14 by way of a fluid flow path defined by the separation element (i.e., rather than the first passage 51 communicating directly with the second passage 52).

In some applications, it is important that a sample of particles present at the inlet region 15 enter each of multiple stepped passageways at substantially the same time. If a device such as that depicted in FIG. 2 is used, it is apparent that particles provided to the inlet region 15 by way of the inlet port 16 will arrive at the outermost stepped passageways (left-most and right-most passages in FIG. 2A) later than they will arrive at the stepped passageway nearest the inlet port 16 (center passage in FIG. 2A). With reference to the device depicted in FIG. 2, the separation element 14 can define walls or channels that originate at the inlet port 16 and extend by various paths to each of the individual stepped passageways, such that the linear flow distance along each flow path is equal. Thus, the flow path extending between the inlet port 16 and the central flow path will be curved, angled, or serpentine relative to the flow paths extending between the inlet port 16 and the outermost flow paths. The end result is that, because the linear flow paths are of equal lengths, particles provided to the inlet port end of each of the flow paths will arrive at the stepped passageway end of the flow paths at substantially the same time.

The separation element 14 includes at least two steps, including a first step 61 nearer (along the stepped passageway) the inlet region 15 than a second step 62. Particles suspended in a fluid flow through the stepped passageway that includes a first passage 51 and a second passage 52 that has a smaller narrow dimension than the first passage 51. Most or all particles in the fluid are able to flow into the first passage 51, but only some of the particles are able to flow through the second passage 52. The net result is that some particles in the fluid can flow through the entire stepped passageway, while other particles are retained within the apparatus, such as within the first passage 51. Segregation of particles is thus achieved.

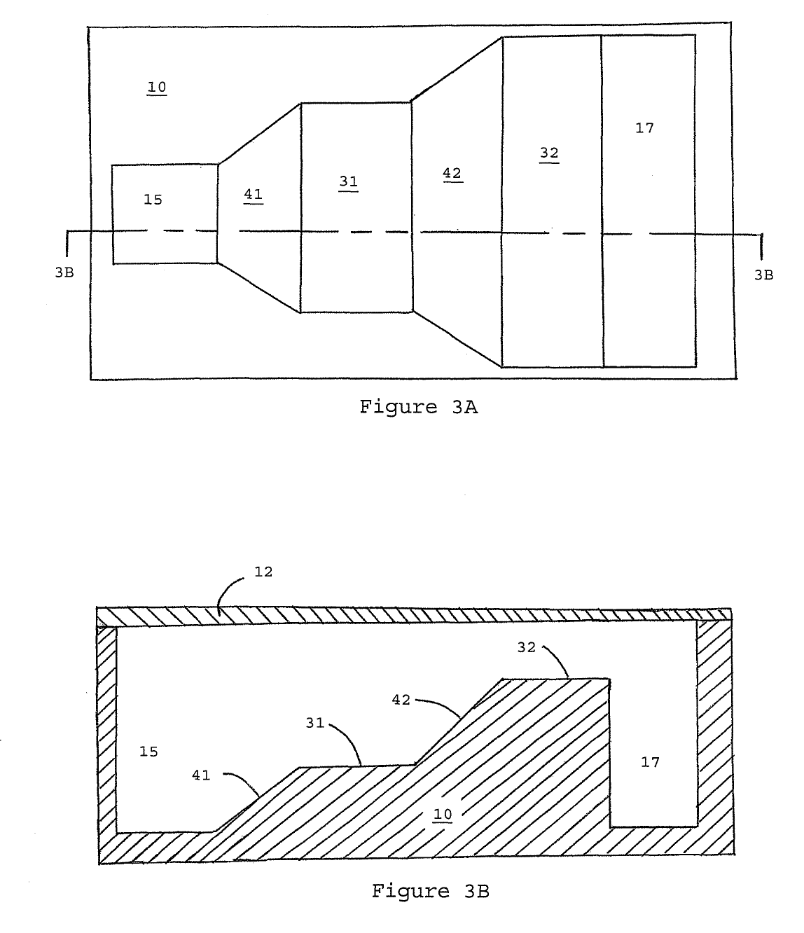

The steps of the separation element 14 can have any of a variety of shapes. In one embodiment (e.g., in the apparatus depicted in FIG. 1), the first step 61 and the second step 62 have a traditional ‘staircase’ step structure, i.e., two planar surfaces that intersect at a right angle. That is, the transitional face 41 of the first step 61 and the broad face 31 of the first step 61 meet at a right angle, as do the transitional face 42 of the second step 62 and the broad face 32 of the second step 62. Alternatively, the transitional and broad faces of the steps can meet at an angle between 90 and 180 degrees, as depicted in FIG. 3, for example. The transitional and broad faces of the steps can also meet at an angle between 0 and 90 degrees, forming an overhang.

Steps that form an overhang and steps that have faces that meet at angles near 90 degrees can induce turbulent flow near the edge at which the faces of the step meet. Such turbulence can dislodge particles that might otherwise occlude the passage between the broad face of the step and the opposed face of the body 10 or cover 12, and this turbulence can thereby inhibit clogging of the passage and enhance fluid flow (and reduce fluid pressure drop) through the device, which are beneficial effects. Furthermore, when the step forms an overhang and the height of the step is sufficiently large that particles that might otherwise clog the passage can reside in the recess formed by the overhang, such steps can also reduce clogging of the passage and improve performance of the apparatus. To the extent that the approximate size of relatively large, undesired particles in a sample can be predicted, one or more steps designed to capture or exclude such particles can be incorporated into the device in order to capture the undesired particles in a place and quantity that does not significantly inhibit fluid flow through the stepped passageway.

Steps having transitional and broad faces that meet at an angle between 90 and 180 degrees can occlude passage of particles having a variety of sizes (i.e., those having sizes intermediate between the narrow dimension of the passage defined by the broad face of the step and the narrow dimension of the space upstream from the step. By halting passage of particles having slightly different sizes at different positions on the transitional face of the step, a step having transitional and broad faces that meet at an angle between 90 and 180 degrees can prevent clogging of the passage defined by the broad face of the step to a greater degree than a step having transitional and broad faces that meet at an angle of 90 degrees or less.

Clogging of fluid flow past a step by particles that occlude the passage defined by the broad face of the step can also be reduced or avoided by increasing the width of the step. Because each particle occludes fluid flow only for the flow area obscured by the particle, a wider step will necessarily be clogged by a greater number of occluding particles. The width of a step can be increased in either or both of two ways. First, the width of the step can be increased by simply increasing the linear width (as depicted in FIG. 4) of the step. Second, the width of the step can be increased by increasing the length of the edge at which the broad and transitional faces of the step meet by decreasing the linearity (i.e., straightness) of the step.

By way of example, in a fluid channel having a rectangular cross-section, a step that extends directly across (i.e., at right angles to the sides) of the channel has an upstream-most edge with an edge length simply equal to the width of the channel. If the shape of the step is a semicircle, with the arc of the semicircle extending such that the center of the semicircle lies downstream from the upstream-most edge of the semicircle, the edge length of the step is equal to the length of the semicircle, which is the number pi multiplied by the width of the channel and divided by two (i.e., roughly 1.57× the width of the channel). Similarly, steps having edges shaped like an arc of a circle or ellipse, like chevrons (i.e., like the letter V), like zig-zags, like serpentine lines, or like irregular lines will all have edge lengths greater than the edge length of a step that extends perpendicularly across a fluid channel having a rectangular cross-section. Steps having edges with such shapes can be used in the apparatus described herein.

The dimensions of the first step 61 and the second step 62 are not critical, except that the second step 62 defines a boundary of the second passage 52, which serves to segregate particles as described herein. For that reason, the dimensions of the second step 62 and the corresponding second passage 52 defined by the second step 62 of the separation element 14 and the opposed surface(s) of the body 10 or cover 12 should be carefully selected. Criteria relevant to selecting these dimensions include the dimensions of the particles to be segregated by their ability to traverse the second passage 52.

By way of example, if relatively large cells are to be segregated from a population of cells of mixed sizes, the narrow dimension of the second passage 52 should be selected such that the relatively large cells are substantially unable to enter the second passage 52 and that other cells in the population are able to enter and traverse the second passage 52. In this instance, the shape and width of the second step 62 should be selected based on the number of relatively large cells that are anticipated to be present in the sample, so that clogging of the second passage 52 by the relatively large cells can be reduced, delayed, or avoided.

Similarly, if particles of limited fluidity (i.e., relatively non-deformable particles) are to be segregated from similarly-sized particles of greater fluidity (i.e., relatively deformable particles), then the narrow dimension of the second passage 52 should be selected to closely match the size of the two types of particles, it being understood that although both types of particles will be able to enter the second passage 52, the relatively deformable particles will, on average, be able to traverse the second passage 52 in less time than the particles of limited fluidity. In this example, it can be advantageous to include a plurality of second passages 52, each having a width and shape sufficient to accommodate the anticipated number of particles without significantly clogging. In this example, it can also be advantageous for each second passage 52 to have a relatively short length, so as to minimize clogging by the relatively deformable particles, which will traverse the second passages 52 in less time than the particles of limited fluidity.

The width (i.e., as defined herein and shown in FIG. 4) of the each of the first step 61 and the second step 62 can be selected based on the anticipated accumulation of particles on the step, in view of the sample anticipated to be processed using the apparatus. Based on the narrow dimension of the second passage 52, the proportion and number of particles that will be unable to enter the second passage 52 can be estimated. Combining this information with the average size of the particles unable to enter the second passage 52 can yield an estimate of the total length-of-step that is likely to be occluded by the particles unable to enter the second passage 52, and that estimate can be used to select an appropriate step width. The width of each step is preferably selected to prevent total occlusion of flow past the step. The width of a step (and the corresponding passage defined by the step) can be selected to be significantly (e.g., 10, 1000, or 100000 times) greater than the narrow dimension of the passage. By way of example, for segregation of fetal-like cells from maternal blood, a step width approximately at least 1000 (one thousand), and preferably 10000 (ten thousand), times the narrow dimension of the corresponding passage is considered desirable. Relatively wide steps permit accumulation of particles within a passage while limiting clogging of the passage.

In some instances, it is desirable to select a narrow dimension of the first passage 51 such that particles unable to enter the second passage 52 will form a layer not more than one particle deep (i.e., in the direction of the narrow dimension of the first passage 51). The width and length of the first step 61 can be selected to accommodate the anticipated number of such cells.

The length (i.e., as defined herein and shown in FIG. 4) of the first and second steps of the separation element 14 are generally not critical, as it is the narrow dimension of the first and second passages (which are bounded by the first and second steps, respectively) that provide the segregative functionality of the apparatus described herein. In situations in which it is desired to accumulate or observe particles on a step, the length of the step can be selected to accommodate the anticipated or estimated number and size of the particles on the step. In instances in which the segregative ability of the apparatus depends on the difference in the relative rates at which particles of different types can traverse one or both of the first passage 51 and the second passage 52, the length of the step can influence the degree of segregation achieved, longer steps enhancing the segregation effected by differing rates of traversal. Step length can be increased by increasing the length of a single step, by increasing the number of steps of a selected length (each step defining a passage having the same narrow dimension), or by a combination of these.

In some embodiments, planar step regions can be substantially parallel to a portion of the cover, a portion of the body, or both, and should have a length (in the direction of bulk fluid flow) equal to a multiple (e.g., 2, 4, 10, or 1000) of the narrow dimension of the passage it bounds. The width of the planar region (in the direction perpendicular to bulk fluid flow) should be equal to a multiple (e.g., 10, 1000, of 10000) of the narrow dimension of the passage it bounds. In some examples of embodiments of the devices described herein, the ratio of the width of the planar region (in the direction of flow perpendicular to bulk fluid flow) ranges from 1,318 at the most open end to 805 at the narrowest (outlet) end; 659 at the most open end to 967 at the narrowest (outlet) end, 537 at the most open end to 725 at the narrowest (outlet) end for each of three separate cassette designs. Gradations on each of the chips increases the ratio of step width to height by 66.7 going from the inlet to the outlet side of the cassette. This width to height ratio will vary depending upon the ratio of the number of particles it is desired to capture within the cassette to those which it is desired to pass through the cassette. As described in Example 4 herein, the ratio of fetal cells to (white blood cells+red blood cells) that are captured by devices of the type described herein can be quite high, and selection of appropriate step height and length can permit passage of greater than 99.99% passage of all nucleated blood cells in a maternal blood sample.

Although the apparatus has been described herein with reference to a first step 61 and a second step 62, additional steps (e.g., three, four, ten, or one hundred steps) can be included in the apparatus, each step defining a passage within the stepped passageway having a characteristic narrow dimension.

The apparatus can include a single separation element 14 or a plurality of separation elements 14. By way of example, the apparatus can include a first separation element that defines a first step 61 and a second separation element that defines a second step 62. If integral with the body 10, the first and second separation elements 14 can be disposed at different locations on the body 10, so long as both separation elements 14 are within the void 11, interposed between the inlet region 15 and the outlet region 17 of the void 11, and define steps in the same stepped passageway. Alternatively, a separation element defining the first step 61 can be integral (or attached to) with the body 10, and a second separation element defining the second step 62 can be integral with (or attached to) the cover 12, so long as both separation elements are within the void 11, interposed between the inlet region 15 and the outlet region 17 of the void 11, and define steps in the same stepped passageway. Similarly, the two separation elements can be discrete pieces, provided the same conditions are satisfied.

The separation element 14 can be constructed from a unitary piece of material (and can be integral with one of the body 10 and cover 12) or it can be constructed from a plurality of pieces of material. By way of example, the separation element 14 of an apparatus like the one depicted in FIG. 1 can be formed of two rectangular bars (solid forms having three pairs of parallel faces, each pair being oriented at right angles to the other two pairs) of material, one bar lying atop a flat portion of the body 10 in the void 11 and forming the first step 61, and the second bar lying atop the first bar and forming the second step 62.

Passage Geometry

The geometry of each step should be selected such that at least some particles will be able to pass through the passage defined by that step, and at least some other particles will not be able to pass through the passage defined by that step. A rigid particle's ability to pass through a passage depends on the characteristic dimensions of the particle. A rigid particle cannot pass through a passage that has a height which is less than the short dimension of the particle. A rigid particle will be substantially uninhibited from passing through a passage that has a height which is greater than the long dimension of the particle. A rigid particle can pass through a passage that has a height which is greater than its short dimension but less than its long dimension, but the passage will at least somewhat inhibit the particle from passing.

The ability of deformable particles (e.g., biological cells, gas bubbles, or cereal grains) to traverse a passage can depend, like the ability of a rigid particle, on its characteristic dimensions. In addition, deformable particles can traverse passages having narrow dimensions smaller than the short dimension of the particle, to the extent the particle can deform to ‘squeeze’ through the passage. This ability depends on the rigidity of the particle, the size of the passage, and the fluid pressure applied against the particle. Where these quantities are not known or predictable, empirical data can be gathered to determine or estimate the ability of such particles to traverse a passage of a given size, and such empirical data can be used to select appropriate dimensions for the first and second passages of the apparatus described herein.

In several parts of this disclosure, reference is made by example to fluid passages having rectangular cross-sections (such cross sections taken perpendicular to the direction of bulk fluid flow). The fluid passages of the apparatus described herein are not limited to such rectangular channels. The walls of the fluid passages can be perpendicular to one another and to one or more of the body 10, cover 12, and separation element 14. The walls can have other arrangements as well. In one embodiment, the fluid passages are rounded, such as passages formed by removal of material by a spinning bit having a rounded tip. Similarly, fluid passages can be rounded on one side (e.g., where formed into the body 10) and flat on another side (e.g., where bounded by a flat cover 12).

Reduction of Shear Stresses

Fluid shear stresses can harm deformable or breakable particles, such as biological cells. Reduction of fluid shear stresses within the apparatus is therefore desirable when the apparatus is to be used to process such particles. Significant fluid shear stress can occur at positions in fluid channels at which the linear flow velocity changes rapidly, such as at locations at which the geometry of the fluid channel changes. The geometry of the fluid channels can be selected to increase, decrease, or maintain constant the linear flow velocity within the apparatus. Increasing or decreasing linear flow velocity creates fluid shear stress. The level of fluid shear stress can be selected to rupture, deform, or destroy some kinds of particles over other kinds of particles. For example, durable particles can be segregated from breakable particles having the same size by inducing fluid shear stress that ruptures the breakable particles. The durable particles are retained in the passageway while the fragments of the breakable particles pass the second step 62 and flow into the outlet region 17. Similarly, substantially constant linear fluid velocity can be maintained throughout the apparatus (or at least throughout the stepped passageway thereof) by selection of appropriate fluid channel dimensions.

The body 10, cover 12, and separation element 14 can be formed such that the cross-sectional area of the stepped passageway with respect to the direction of fluid flow increases, decreases, or remains constant. The cross-sectional area of the stepped passageway affects the pressure and flow rate of the fluid in the apparatus. If the separation element has a constant width, then the cross-sectional area defined by the height and width of the first passage 51 will be smaller than the cross-sectional area of the inlet region 15. The cross-sectional area of the second passage 52 (e.g., defined by the height and width of the second passage if it is rectangular in cross section) will be smaller than that of the first passage 51. As the cross-sectional areas of the passages decrease, the fluid pressure and flow rate of fluid flowing through the cross-sectional areas increases. The geometry of the fluid channels can be selected to counteract these changes in fluid pressure and flow rate. For example, the width of a passage having a rectangular cross section can increase proportionally as the height of the passage decreases, such that the cross-sectional area of passage is constant. For a separation element 14 where each step is separated by sloped transition face, the width of the passage defined by the transition face can increase at a constant rate, equal to the rate at which the height of the passage decreases. The fluid pressure and flow rate through the passageway defined by such a separation element remains constant. An example of such a passageway is shown in FIG. 3.

Put another way, the body 10, cover 12, and separation element 14 can be formed such that fluid flux is equal at all places throughout the narrow passageway of the apparatus. For example, in the apparatus shown in FIG. 3, fluid flux throughout the inlet region 15, the passages defined by surfaces 41, 31, 42, and 32, and the outlet region 17 can be constant. Alternatively, the body 10, cover 12, and separation element 14 can be formed such that fluid flux increases or decreases in the direction of bulk fluid flow. For example, the surfaces of the body 10 or cover 12 that define the width of the void 11 can taper in the direction of the inlet region 15 or outlet region 17.

Fluid shear stresses are, of course, not a concern when the apparatus is operated without a fluid in the stepped passageway. Because the viscosities of gaseous fluids are substantially lower than the viscosities of liquid fluids, fluid shear stresses are of lesser concern when the particles are suspended in a gaseous fluid (e.g., air) than in a liquid fluid. Similarly, because fluid shear stresses vary in known ways with fluid viscosity, modifications of the apparatus described herein suitable for accommodating fluids of different viscosities will be apparent to the ordinarily skilled designer.

The body, the cover, or both, can have one or more fluid channels that fluidly connect with the surface of a step of the separation element, for removing fluid from the step (including any cells suspended in the fluid upon that step). Furthermore, when the step has regions or discrete grooves in the step, the cover or body can be machined so that the fluid channels fluidly communicate most nearly with a discrete groove or region upon the step, for removing fluid in the vicinity of that groove or region of the step. Such local channels can improve purification by capturing only a relatively small amount of fluid in the immediate vicinity of the channel when a particle is captured thereby. Likewise, the body, the cover, the separation element, or some combination of these, can have an optical, electrical, or optico-electrical device constructed therein or thereon (e.g., by etching, film deposition, or other known techniques) at a position that corresponds to a selected step or a selected groove or region of a step. Such devices can be used to detect cells (e.g., using a detector to detect a decrease in light or other radiation transmitted across the fluid between the surface of the step and the cover or body) or to manipulate cells (e.g., using an activatable heating element to ablate cells which pass or rest near the heating element). Devices constructed upon the cover, the body, or the steps can be made individually activatable by assigning an electronic address to the device. In this manner, cells can be detected at discrete areas of the device, and cells at selected areas can be manipulated without manipulating cells at other positions.

Harvesting of cells from a selected step (or a plurality of selected steps) can be performed by simply withdrawing fluid from that step or a portion of the step. In some instances, such as when adhesion between cells and a step upon which they rest occurs, it can be advantageous to apply energy to the apparatus in order to dislodge the cells or otherwise facilitate their removal. The energy can be applied in many forms, and a preferable form will usually depend on the type of cell or object to be displaced and the identity of the force or phenomenon which inhibits removal of the cell or object from the step. By way of example, withdrawal of fluid from one portion of a step can be performed simultaneously with addition of fluid at another portion of the same step. Other examples of forms in which energy can be applied to the apparatus in order to harvest cells include shaking, tapping, or vibrating the apparatus, or applying energy in the form of ultrasound, heat, infrared or other radiation, bubbles, compressed air, and the like.

Instead of recovering cells that are retained on one or more steps of the separation element, the cells can instead be detected or manipulated. In one embodiment, one or more cells are lysed by application to the cells of electrical, mechanical, or heat energy, thereby releasing the contents of the cell in the void of the apparatus. The cell contents can be analyzed or manipulated in the apparatus, or they can be recovered from the apparatus and analyzed or manipulated outside of the apparatus. By way of example, a cell that is retained at a particular location on a step can be lysed using a device located at or focused upon that particular location, thereby releasing the cell's DNA into the void. The DNA can be amplified in the void by providing PCR reagents to the void, or it can be collected (e.g., in a container in which fluid obtained from a selected portion of the void is collected or, alternatively, by passing fluid through the void and collecting the DNA in the outlet fluid) and amplified outside of the apparatus. The apparatus can thus be used to analyze the contents of individual cells or groups of cells.

Any of a wide variety of methods for harvesting or manipulating cells within a device can be employed using the apparatus described herein. By way of example, methods employing known “optical tweezer” devices, laser microdissection devices, and particle-binding membranes and films can be employed. In embodiments employing a film or membrane, the film or membrane can overlie an orifice or fluid channel, sealing the orifice or fluid channel from the remainder of the void. Upon observation that a particle of interest is adhered to or rests upon the film or membrane, the portion of the film or membrane contacting the particle (or an area surrounding the particle) can be detached or punctured, placing the particle in fluid communication with the orifice or fluid channel previously segregated by the film or membrane. If the film or membrane has an optical, magnetic property by which it can be identified, then the detached portion of the film or membrane (e.g., having a particle of interest attached thereto) can be isolated either by screening for a characteristic of the particle or for a characteristic (e.g., a spectrophotometric property or magnetic property) of the film or membrane. Furthermore, if the film or membrane has a property (e.g., magnetism) by which the film can be urged to move in a selected direction, the film can be used to mechanically manipulate particles attached to it. For example, a detached portion of a magnetic film or membrane having a cell attached to it can be used as a transportation vehicle for that cell by applying a directional magnetic field to a fluid in which the membrane is suspended or by moving a magnetic probe to guide the detached portion of the magnetic film or membrane with cell attached to it towards a desired location such as a channel, chamber or container.

The Membrane or Other Barrier

The apparatus can include a membrane or other barrier 81 that is in fluid communication with a portion of the void 11. The membrane or other barrier 81 is selected such that it preferentially segregates desired particles contacting it in the void from undesired particles. In one embodiment, the membrane or other barrier 81 is a membrane having pores of a defined size, such that particles smaller than the pores can pass through the membrane (e.g., when fluid flow or a pressure differential occurs across the membrane) while particles larger than the pores are prevented from passing through the membrane. Alternatively, the membrane or other barrier 81 can be a porous membrane made of a hydrophobic material, such that relatively hydrophilic particles will tend to be repelled from the membrane surface (and not pass through its pores) while relatively hydrophobic particles can pass through the membrane's pores. In still another alternative, the membrane or other barrier 81 is a barrier (porous or, preferably, non-porous) made from a material through which desired particles can pass, but non-desired particles cannot pass.

By way of example, fetal cytotrophoblasts are able to pass through maternal uterine tissue, including extracellular matrix of the uterus. Such cells, contained in the void 11 of the apparatus disclosed herein and brought into contact with a barrier made of uterine membrane or a similar material (e.g., basement membrane extract such as the Matrigel line of basement membrane matrix products available from BD Biosciences, San Jose, Calif.) are able to burrow into and penetrate through a relatively thin barrier of such material. Few, if any other cell types are known to be capable of penetrating such matrix.

For example, fetal cytotrophoblasts in maternal blood can be segregated as described elsewhere herein by virtue of their inability to pass through regions of the void 11 of the apparatus described herein. Other cells present in maternal blood may also be unable to pass through the same regions of the void 11, and may remain with the fetal cytotrophoblasts within the apparatus. If this population of cells is contacted with a thin, solid barrier made of a Matrigel™ type material, the fetal trophoblasts will be able to adhere to, enter, and penetrate the barrier, and can be segregated from other cells of the population that are unable to adhere to, enter, or penetrate the barrier. In this way, segregation of fetal cytotrophoblasts from other cells and materials present in maternal blood can be effected based on two properties of the cells and materials (i.e., ability to pass through the apparatus described herein and interactions with a barrier of a basement membrane-like matrix barrier). Likewise, such an apparatus can be used to segregate fetal cytotrophoblasts that are able to interact with or penetrate through such a barrier from fetal cytotrophoblasts that have lost (or never had) such abilities.

The membrane or other barrier 81 can be a part of the apparatus described herein, combined with the apparatus after particle segregation is performed using the apparatus, or used separately from the apparatus before or after particle segregation is performed using the apparatus. In a preferred embodiment, the membrane or other barrier 81 is situated adjoining a portion of the void 11 in which desired particles are anticipated to remain following particle segregation performed using the apparatus. In this embodiment, a first population of particles can be segregated as described herein, generating a second population of particles (e.g., particles able to traverse the first passage 51 but unable to enter the second passage 52) that includes desired particles. If the membrane or other barrier 81 is situated adjoining a portion of the void 11 that contains the second population of particles, then those particles can be segregated by virtue of their differential ability to interact with the membrane or other barrier 81. By way of example, if the membrane or other barrier 81 includes a material (e.g., a monoclonal antibody) that specifically binds with a subset of particles in the second population, then that subset can be segregated from other particles in the population. Similarly by way of example, if a subset of particles in the second population is able to pass through the membrane or other barrier 81, then that subset can be segregated from other particles in the second population that are not capable of passing through the membrane or other barrier 81.

When a face of the membrane or other barrier 81 adjoins a portion of the void 11, the opposite face of the membrane or other barrier 81 can adjoin a second void or a second material. The second void or second material can, optionally, include one or more ingredients that interact with (e.g., bind to or are consumed by) particles that cross the membrane or other barrier 81. By way of example, in an apparatus for segregating populations of cells, the apparatus can include a semi-permeable membrane that selectively permits a subset of cells to pass therethrough, with one face of the membrane adjoining the void 11 of the apparatus at a location where cells of the subset are expected to occur and the other face of the membrane adjoining a second void that contains a growth medium for supporting metabolism and/or proliferation of cells. Alternatively, or in addition, the second void can contain a second material that specifically binds with cells of a selected type, such that when cells of the selected type cross the membrane, they bind with the second material and tend not to re-cross the membrane. Passage of cells or other particles across the membrane or other barrier 81 can be mediated by fluid flow across the membrane or other barrier 81, by motility of the cells or particles, by gravity, by diffusion, or otherwise.

Materials and Methods of Construction

The identity of material(s) used to construct the body 10 and the cover 12 are not critical, except that they should be sufficiently rigid that the parts will maintain their shapes, and not substantially deform or break, during operation of the apparatus as described herein. Where deformable materials are used, the expected deformation under conditions of operation should be taken into account when designing the size and shapes of the parts. Examples of suitable materials include glasses, solid polymers such as polytetrafluoro-ethylenes and epoxy resins, and crystalline minerals such as silicon. The body 10, cover 12, separation element 14, and other components described herein can each be formed from a different material, if desired. Preferably, all parts are formed of the same material, so that the effects of, for example, temperature, on expansion and contraction of parts is similar for all parts.

It can be beneficial to observe the movement, status, or behavior of particles in the apparatus. In such instances, at least one of the body 10 and the cover 12 should be constructed from a material that facilitates observation of the particles in the assembled apparatus. By way of example, many glasses are transparent to wavelengths of light in the region of the optical spectrum that is visible to the human eye. Construction of one or more parts of the apparatus from such a glass permits an operator to visually inspect particles in the void (e.g., accumulation of particles in the first passage 51) during operation of the apparatus.

The identity of the materials used to construct the separation element 14 is also not critical, except that it should be sufficiently rigid that the separation element 14 will maintain its shape, and not substantially deform or break, during operation of the apparatus as described herein.

Selection of materials used to construct the apparatus and its parts can be influenced by the nature of the particles to be segregated therein. The nature of the particles can also influence decisions regarding which, if any, surface treatments may be appropriate for modulating interaction of particles with surfaces they may encounter within the device. For example, if particles are to be segregated within the device without substantially binding or adhering to the device, then the materials and/or surface treatments should be selected to reduce or eliminate the likelihood of particle binding to the surfaces. Alternatively, one or more surfaces of the device (e.g., the broad surface 31 of the first step 61) can be treated in such a way that particles (or particular types of particles within a mixed population of particles) will adhere to or bind with the surface(s). By way of example, biological cells are known to express a variety of proteins on their surface, and antibodies that specifically bind to a protein of a selected type can be generated by known methods. If antibodies that specifically bind to a protein expressed on the surface of cells of a particular type are fixed to a surface in the stepped passageway, binding of the cells of the particular type with the antibodies can be expected to inhibit or halt passage of the cells past the surface in the apparatus, enhancing the segregation of those cells from cells that do not express the protein on their surface (and to which the antibodies cannot bind).

Selection of methods to construct the apparatus can be influenced by the size of the particles to be separated therein. The particular method employed to construct the apparatus and its parts is not critical. A wide variety of methods of forming parts having shapes and conformations that are accurate to the micrometer and nanometer scale are known. For example, any of a variety of known micromachining methods can be used. Examples of such micromachining methods include film deposition processes, such as spin coating and chemical vapor deposition, laser fabrication, and photolithographic techniques such as UV or x-ray processes, precision machining methods, or etching methods which may be performed by either wet chemical processes or plasma processes. (See, e.g., Manz et al., 1991, Trends in Analytical Chemistry, 10:144-149). Alternatively, the parts can be molded, rather than machined, using any of a variety of known molding methods. A wide variety of methods of forming and machining parts for use on a macroscopic scale are known, such as cutting, carving, molding, engraving, welding, and casting.

The body 10, cover 12, and separation element 14 can be constructed separately and assembled to form the apparatus, and such assembly can be performed by the manufacturer or the user of the apparatus. Alternatively, the separation element 14 can be constructed as an integral part of one of the cover 12 or the body 10. In one embodiment, a single cover 12 is made capable of sealing a void 11 formed with any of a variety of bodies 10 (e.g., each having a separation element 14 in the void 11 of the body 10, the various separation elements 14 having different properties, such as different step heights).

Segregable Particles

The apparatus segregates particles based on the ability of various particles to traverse the first and second passages of the apparatus described herein. The particles that can be segregated using the apparatus include living particles such as animal or plant cells, bacteria, or protozoa, or non-living particles. The apparatus described herein can be used to segregate larger particles (e.g., cereal grains, rodent feces, gas bubbles, and bowling balls) and smaller particles (e.g., subcellular organelles, viruses, and precipitated mineral particles).

Attributes of the particles that affect their ability to traverse the first and second passages of the apparatus described herein include the size, shape, surface properties, and deformability of the particles.

A particle tumbling randomly in a fluid will sweep out an exclusion volume equal to the volume of a sphere having a diameter equal to the longest dimension of the particle. Thus a rigid sphere having a diameter of 1 micrometer, a randomly-tumbling disk-shaped rigid particle having a diameter of 1 micrometer and a thickness of 0.2 micrometers, and a randomly-tumbling rod-shaped rigid particle having a length of 1 micrometer and a diameter of 0.1 micrometer will each sweep out an equal exclusion volume. Ignoring the effects of surface properties, each of these particles will be able to traverse a passage having a narrow diameter greater than 1 micrometer. The disk-shaped and rod-shaped particles will be able to traverse a passage having a narrow diameter less than 1 micrometer and greater than 0.2 micrometer. The rod-shaped particles will be able to traverse a passage having a narrow diameter less than 0.2 micrometer and greater than 0.1 micrometer. The ability of non-rigid (i.e., deformable) analogs of these particles to traverse one of these passages (and the rate at which such traversal can occur) depends on the degree and extent of deformability of the particles and the extent to which the particles need deform in order to fit within the passage. Furthermore, the surface properties of the particles and the surfaces that define the passage can affect the rate at which the particles traverse the passage, and can prevent such traversal from occurring (e.g., if the particle binds avidly with the surface of the passage or if the surfaces of the passage and the particle repel one another).

In important embodiments, the particles that are separated are biological cells present in a mixed population of cells (i.e., a suspension of cells that include cells of multiple types). Selection of appropriate narrow dimensions for the first and second passages of the apparatus described herein permits segregation of biological cells based on their size, shape, surface properties, deformability, or some combination of these properties. Examples of biological cells that can be separated using the apparatus described herein include fetal cells circulating in maternal blood, embryonic stem cells (in maternal blood or an individual's own embryonic stem cells), adult stem cells, tumor cells, bacteria and other pathogens, and cells of the immune system (e.g., various white blood cells such as T cells, B cells, neutrophils, macrophages, and monocytes). The methods can be used to segregate mixtures of cells of these types. The methods described herein can be used to segregate sub-cellular organelles (e.g., nuclei, chloroplasts, and mitochondria) as well.

In another important embodiment, the apparatus is used to isolate agents of infectious diseases (e.g., bacteria or viruses) or other pathogens (e.g., protozoa or parasites) from a sample. In these embodiments, the apparatus can be used for diagnostic purposes, such as analyzing a biological sample obtained from a subject in order to determine whether the subject is infected with an infectious agent. In another example of these embodiments, a sample such as a water sample or a food product or ingredient can be assessed by using an apparatus described herein to assess the sample directly, or a fluid with which the sample is contacted, for the presence of a pathogen which, if ingested by a subject, would contribute to the likelihood that the subject would develop a disease or other condition.

For example, stem cells can be segregated from other cells present in maternal blood or in placental blood. Such blood includes a variety of cells, including stem cells, red blood cells, and platelets. Blood is preferably collected upstream of capillary beds when the cells that are sought have a size (i.e., diameter>8-10 micrometers) exceeding the normal diameter of capillaries. Thus, arterial blood (e.g., blood taken from the common hepatic artery) or a fluid derived from such pre-capillary blood (e.g., lung and bronchial exudates and secretions, or fluids containing them, such as bronchial lavage fluids) is a preferred source for large cells such as fetal trophoblasts and stem cells. Human stem cells tend to exhibit an exclusion volume equal to a sphere having a diameter of about 12 micrometers. Human red blood cells tend to exhibit an exclusion volume equal to a sphere having a diameter of about 5.5 micrometers. Human platelets tend to exhibit an exclusion volume equal to a sphere having a diameter of about 1 micrometer. Ignoring deformability and surface property effects, the stem cells, but not the red blood cells or platelets will be excluded from a passage having a narrow dimension on the order of 4 to 8 micrometers. Stem cells provided to the inlet region 15 of an apparatus described herein with a second passage 52 having a narrow dimension of 4 to 8 micrometers will generally not pass to the outlet region 17 of the apparatus, although red blood cells and platelets will. If the narrow dimension of the first passage 51 is greater than about 12 micrometers (e.g., if the narrow dimension of the first passage 51 is 18 micrometers), then stem cells, red blood cells, and platelets will all traverse the first passage 51. If maternal or placental blood is provided to the inlet region 15 of an apparatus described herein with a first passage 51 having a narrow dimension of 18 micrometers and with a second passage 52 having a narrow dimension of <8 micrometers, and the blood is passed through stepped passageway of the apparatus, then red blood cells and platelets will pass through (i.e., through the first and second passages to the outlet region 17 of) the apparatus, while stem cells will be retained upstream from the second passage 52. If an apparatus configured such as the one depicted in FIG. 1 is used (i.e., wherein there is no intervening passage or chamber between the first and second passages), the stem cells will accumulate in the first passage 51. Passage of additional cell-free fluid through the apparatus following passage of the blood will tend to increase the proportion of red blood cells and platelets that are segregated from the stem cells. Backflushing of the apparatus (i.e., with fluid flow occurring in the direction from the outlet region 17, through the stepped passageway, toward the inlet region 15) can flush the stem cells from the apparatus into the inlet region 15, whence they can be recovered.

Particles within the stepped passageway are subjected to shear, compressive, and other forces acting upon them by any fluid flowing through the passageway. If particles (e.g., biological cells) that exhibit different resistant to deformation, compression, bursting, lysis, or breakage (i.e., any characteristic that alters the rate or ability of the particle to traverse one or both of the first and second passages) are present, the differences in response of the particles to fluid flow can be used to differentially affect passage (or non-passage) of the particles through the stepped passageway. By way of example, in a mixture of cell types including cells that lyse readily under fluid shear and cells of substantially the same size that do not substantially lyse under fluid shear, these two types of cells can be separated from other particles under conditions of relatively low fluid flow (i.e., flow low enough that few or no cells lyse). After such separation, the fluid flow rate can be increased in order to generate sufficient fluid shear within at least one portion of the stepped passageway that cells of the first type, but not cells of the second type, will lyse, yielding first cell type lysis products in the effluent from the outlet region and cells of the second type retained within the apparatus.

Fluid Displacement Devices

The apparatus described herein can be operated by providing particles to the inlet region 15 of the void 11 of the apparatus and permitting the particles to move through fluid present in the inlet region 15, the stepped passageway, and the outlet region 17, such movement being attributable to intrinsic motility of the cells or to passive settling of non-motile particles under the influence of gravity. In the latter instance, the apparatus will need to be oriented such that gravity will tend to cause particles that are denser than the fluid to ‘fall’ from the inlet region 15, through the stepped passageway, and toward the outlet region 17 or, for particles that are less dense than the fluid, to cause the particles to ‘rise’ from the inlet region 15, through the stepped passageway, toward the outlet region 17.

More typically, the apparatus described herein is operated by fluidly connecting a reservoir containing a fluid (e.g., a particle-containing suspension or a particle-free fluid) or another fluid displacement device such as a pump to the inlet region 15. Fluid flow through the apparatus is achieved by introducing fluid at the inlet region 15 of the apparatus, by continuously withdrawing fluid from the outlet region 17 of the apparatus, or both. Fluid introduced at the inlet region 15 displaces fluid already present within the void 11 and induces emission of fluid from within the void 11 into the outlet region 17 or through an outlet port 18 that fluidly communicates with the outlet region 17. As particles traverse the stepped passageway of the apparatus, they will emerge therefrom into the outlet region 17. Such particles can be recovered from fluid that accumulates within the outlet region 17 or a reservoir that fluidly communicates with it or from fluid that is withdrawn from an outlet port 18 that fluidly communicates with the outlet region 17. Particles that are unable to traverse either the first passage 51 or the second passage 52 of the apparatus during fluid flow through the apparatus will be retained within the apparatus and can be recovered therefrom.

The identity of the fluid displacement device that is used to provide fluid flow to the inlet region 15 is not critical. The fluid displacement device can be simply a reservoir containing fluid that is permitted to drain, under the influence of gravity, through the apparatus by way of a fluid connection between the reservoir and an inlet port 16 that fluidly communicates with the inlet region. A mechanical pump can deliver fluid to the inlet port 16 by way of a sealed fluid connection between the pump outlet and the inlet port 16. Fluid delivered by the pump displaces fluid present in the inlet region 15 of the apparatus into the stepped passageway and thence toward the outlet region 17, from which displaced fluid can be withdrawn, collected, or emitted. Alternatively, a mechanical pump can withdraw fluid, by way of a sealed fluid connection, from an outlet port 18 in fluid communication with the outlet region 17 of the apparatus. Withdrawal of fluid from the outlet region 17 lowers the fluid pressure at the outlet region 17, inducing displacement of fluid from the adjoining stepped passageway of the apparatus into the outlet region 17 and from the inlet region 15 into the stepped passageway.

Positive displacement of fluid in the void 11 of the apparatus (e.g., induced by pumping fluid into the inlet region 15) increases fluid pressure within the void. Increased fluid pressure can alter the dimensions of the apparatus (e.g., by inducing flexion or displacement of parts of the apparatus), the dimension of particles within the apparatus (e.g., deformable gas-filled particles will tend to decrease in size as the surrounding fluid pressure increases), or both. Moreover, pulsating or otherwise varying fluid pressure can induce transient changes in localized fluid flow within the apparatus.

Transient localized flow variations can be beneficial. For example, particles which are unable to enter the first or second passage of the stepped passageway can be urged against the upstream extent of the passage, blocking fluid flow through the portion of the passage occluded by the particle. Transient variations in flow of fluid at the point of occlusion of the passage by the particle can alternately urge the particle against the passage opening and urge the particle away from the opening, thereby temporarily relieving the occlusion and permitting fluid flow through the previously-occluded portion of the passage.

Fluid pulsations or other rapid flow changes can induce shear stresses in the fluid and upon particles suspended in the fluid, and particles can be damaged by such shear stresses. Particle damage (e.g., lysis of biological cells) can be reduced by reducing shear stresses within the fluid and their causes. Apart from modifications in the geometry of the fluid channels of the apparatus discussed elsewhere in this disclosure, alterations in the types and characteristics of fluid displacement devices connected with the apparatus can increase or reduce shear stresses within the fluid. By way of example, pumps which deliver fluid at a relatively constant volumetric rate (i.e., rather than a more pulsatile volumetric rate, as with many peristaltic pumps) can reduce fluid shear stresses induced by tidal surges in fluid pressure within the apparatus. Further by way of example, pumps which deliver fluid at a relatively constant pressure (i.e., pumps which monitor the fluid pressure within the output stream of the pump and adjust volumetric flow rate accordingly to maintain a constant pressure) can reduce fluid shear stresses that would otherwise build as portions of the first and/or second passage of the stepped passageway become occluded with particles or debris if volumetric flow rate were not adjusted accordingly. An example of a pump suitable for moving fluid through the apparatus is a low pulse syringe pump. Such a pump can include an agitation mechanism, which may be useful to prevent particles from settling during operation of the apparatus.

Negative displacement of fluid from within the void 11 (e.g., induced by withdrawal of fluid from the outlet region 17) reduces fluid pressure within the void 11 and can induce similar difficulties, including deformation and displacement of parts of the apparatus and transient flow variations. Negative displacement of fluid from the void 11 can also induce bubble formation within fluid in the apparatus, and bubbles can disrupt operation of the apparatus (e.g., by occluding fluid flow through a portion of a passage or by inducing surface tension-related effects upon particles in the apparatus). Bubble formation should therefore be avoided. Positive fluid displacement of fluid within the void 11 of the apparatus is preferred for this reason.

In one variation, fluid is displaced through the apparatus by application of centrifugal “force” to a fluid-containing reservoir in fluid communication with the inlet region 15 of the apparatus. Centrifugal “force” is generated by spinning the reservoir about an axis, and conservation of angular momentum of the fluid urges the fluid away from the axis of rotation. This “force” can be used to displace fluid from the void 11 of the apparatus by fluidly connecting the reservoir outlet with the inlet region 15 of the apparatus. By way of example, an centrifugally-operable apparatus can include, in a linear arrangement from a position proximal to the axis of rotation toward a position distal to the axis of rotation, a fluid reservoir, the inlet region 15 of the void 11, the stepped passageway, and the outlet region 17 of the void 11. Fluid from the reservoir is driven by centrifugal “force” into the inlet region 15, thence through the stepped passageway (the first passage 51 being located proximal to the axis of rotation relative to the second passage 52), and thence to outlet region 17, which can include a second reservoir for collecting fluid that has passed through the apparatus. Particles unable to traverse the second passage 52 will remain within the void 11 after some or all of the fluid in the fluid reservoir has passed through the apparatus.

Confirming Assembly of the Apparatus

In many applications, significant dimensions of fluid channels of the apparatus described herein have relatively limited tolerance. That is, appropriate operation of the apparatus can depend on the fluid channels maintaining dimensions within a relatively narrow range (i.e., on the order of micrometers to tens of nanometers). Because the apparatus includes at least a cover 12 and a body 10 that are assembled to yield an operable device and because, in operation, positive internal fluid pressure is exerted within the apparatus that would tend to separate the cover 12 and body 10, some means of clamping or otherwise holding the body 10 and cover 12 in their assembled position is usually employed. Pressures induced by clamping or otherwise holding the cover 12 and body 10 in their assembled positions can induce deformation of the parts of the cover 12 or the body 10, potentially altering the significant dimensions of the parts. It is important to detect such deformation when it occurs.

The disclosure includes a method of confirming appropriate assembly of the apparatus described herein. This method is exemplified for an apparatus that includes a body 10 that defines a void 11 and a cover 12 that covers the void 11 and has a flat surface opposite the face that covers the void 11. However, substantially the same method can be used to detect deformation in a part for other configurations by including a flat surface on the face of a part in which deformation is to be detected. In order to confirm appropriate assembly of the apparatus, the body 10 and cover 12 are assembled, including all clamps, holders, or other devices that exert pressure upon any portion of the body 10 or cover 12. Optionally, a particle-free fluid is flowed through the apparatus at the operating pressure to be used. The flat surface of the cover 12 is illuminated with radiation. The interference pattern of radiation reflected or refracted by the flat surface of the cover 12 is examined. The interference pattern indicates the location and extent of bending in the cover and permits confirmation, for example, of whether the variation in the distances between the face of the cover 12 that defines the void 11 and the walls of the void 11 defined by the body 10 is within the appropriate tolerance.

The apparatus can include a variety of visual indicators that confirm proper assembly of the apparatus. A visual indicator is a feature of the body or cover that has one appearance when the apparatus is properly assembled, and a different appearance when the apparatus is not properly assembled. Substantially any visually-observable phenomenon can serve as the visual indicator. As indicated above, interference patterns indicating deformation of a part of the apparatus can be used. Alignment of lines drawn, painted, or inscribed on mating parts can serve as a visual indicator of proper assembly.

Using the Apparatus

The apparatus can be used to segregate particles, such as biological cells, that are suspended in a fluid sample. The fluid sample is introduced at the inlet region 15 of the void 11. Particles in the sample move from the inlet region 15 into a stepped passageway defined by the separation element 14 and at least one of the body 10 and the cover 12. Movement of the particles within the apparatus occurs by virtue of inherent motility of the particles (e.g., for motile biological cells), by density-mediated settling or rising of particles through the fluid within the apparatus, or in response to bulk fluid flow that is induced within the apparatus. The stepped passageway includes a first passage 51 that is bounded by a first step 61 of the separation element 14. The first passage 51 has a narrow dimension (i.e., the distance between the surface of the first step 61 and the opposed face of the body 10 and/or cover 12), and some particles may be unable to enter the first passage 51 on account of their size (taking into account the deformability of the particle). Particles that are able to traverse the first passage 51 continue to move along the stepped passageway to a second passage 52 that is bounded by a second step 62 of the separation element 14. The second passage 52 has a narrow dimension (i.e., the distance between the surface of the second step 62 and the opposed face of the body 10 and/or cover 12) that is narrower than the narrow dimension of the first passage 51, and some particles may be unable to enter the second passage 52 on account of their size (taking into account the deformability of the particle). Particles that are able to traverse both the first passage 51 and the second passage 52 continue to move along the stepped passageway to the outlet region 17 of the void 11. The apparatus thus segregates particles unable to enter the first passage 51, particles able to traverse the first passage 51 but unable to enter the second passage 52, and particles able to traverse both the first passage 51 and the second passage 52. These populations of particles can be separately recovered, as can particles able to enter, but not traverse (during the period of operation) one of the first and second passages. Alternatively or in addition, effluent recovered from the outlet region of the apparatus can be recovered. In one embodiment particles unable to traverse one or both of the first and second passages can be lysed or otherwise degraded (i.e., to permit the lysis or degradation products to pass through the device) prior to recovering the effluent.

Multiple apparatuses can be operated at once (i.e., simultaneously), with the same fluid sample applied to the inlet region 15 of each apparatus. The multiple apparatuses can have a common inlet region 15 or a common upstream reservoir that fluidly communicates with each of the inlet regions 15. It is immaterial whether the multiple parallel apparatuses share the same body 10, the same cover 12, or both. A plurality of discrete apparatuses can be operated independently, of course. In one embodiment, a plurality of apparatus are grouped, bonded, or pressed together to form a mass (e.g., a block of wafers, each wafer acting as a body 10 for one apparatus on one face of the waver and a cover 12 for an adjacent apparatus on the opposite face of the wafer) having the inlet regions 15 (or fluid channels that fluidly communicate with the inlet regions 15) at one end of the mass. A fluid sample including particles can be applied to the end of the mass, and the fluid sample can thereby be provided to the inlet region 15 of each apparatus of the mass. Fluid flow can be induced through all of the apparatuses of the mass by providing fluid to the same end of the mass under pressure (e.g., using a pump). This arrangement allows scale-up of the apparatus and methods described herein without re-engineering or redesign of the components of the apparatus. Instead, the number of wafers can simply be increased to accommodate the anticipated number of particles.

Particles and cells obtained using the apparatus and methods described herein can be used for any of a wide variety of further purposes. Furthermore, for many of those purposes, it is not necessary to isolate particles that may remain within the apparatus after its operation for segregation purposes. By way of example, in many instances, the interaction of intact biological cells or components of biological cells with reagents (e.g., antibodies, enzyme substrates, potentially complementary nucleic acids, and nutrients) can be observed as well for cells that remain within the apparatus as those interactions can be observed for cells recovered from the apparatus. Furthermore, the fluid channels present within the apparatus can facilitate delivery of such reagents to the cells that remain within the apparatus. Thus, the apparatus can be used both to segregate cells and, thereafter, as a reaction vessel to observe interactions of cells with various reagents.

When the apparatus is used to contain fluids that include biological cells, the fluids should preferably be selected to have an osmolarity sufficient to maintain the integrity of the biological cells. If viability or other biological functions of the cells are considered important, then the fluids should also be selected so as to maintain the desired biological function(s).

The apparatus having particles remaining within it can also be used as a container for storing, maintaining, or contacting reagents with the particles. By way of example, the apparatus can be used to segregate within the apparatus bacteria that occur in a sample (e.g., a fluid sample with which a foodstuff such as a chicken egg is washed). After segregating the bacteria within the apparatus, growth media can be provided to the void 11 of the apparatus to encourage survival and multiplication of the bacteria. Indicators (e.g., antibodies that specifically bind a particular bacterial antigen or a reagent that is metabolizable only by harmful bacteria) can be provided to the void and their interaction with the cells therein can be observed. Such an example is useful for analysis of contamination of the foodstuff with pathogenic bacteria.

Age of Blood Samples

While using the apparatus described herein, it was discovered that the flow characteristics of blood and blood cells in the apparatus are significantly altered over time. It is believed that degeneration of blood cells begins soon after the blood sample is drawn, and the effects of the degeneration begin to compromise the effectiveness of the segregation effected by the apparatus disclosed herein after several hours. This may be due, at least in part, to lack of oxygen, nutrients, exposure to fragments of white blood cells that may adhere to the surfaces of the apparatus, or exposure to enzymes released by lysed white blood cells. Blood cells tend to become unstable and are more prone to lysis when passing through the apparatus approximately six to eight hours after the blood sample is drawn. It becomes more difficult to effectively segregate the cells in a blood sample approximately 10-12 hours after the sample is drawn. Blood samples should preferably be used not more than six hours after they are drawn, and not more than about 12 hours thereafter.

In further manipulations of blood samples more than eight hours old, it became apparent that the changes observed in the blood samples were not specific to the apparatus and the method described herein, but are instead a more general phenomena that can be relevant to a wide variety of analyses performed using blood samples. In any analysis that involves passage of blood or blood cells through a relatively narrow passage (i.e., 100 micrometers or less), it appears to be advantageous to perform the analysis using a blood sample obtained from a subject less than twelve hours prior to the analysis, and preferably less than ten, less than eight, or less than seven hours prior to the analysis. Because the apparatus described herein can be operated conveniently by an operator having relatively little expertise, the apparatus can be used to analyze a blood sample at a time very near the time blood is obtained from a subject, such as within a doctor's office or at a phlebotomy laboratory.

Culture and/or Proliferation of Segregated Cells

The methods and apparatus described herein can be used for separation and selective culture of fetal cytotrophoblast to obtain sample for noninvasive prenatal diagnosis.

Fetal trophoblasts can be segregated from other cells of (for example) a maternal blood sample and cultured by first triggering these cells to invade into and migrate through a porous membrane, leaving behind contaminating maternal nucleated cells which are incapable of such invasion. Thereafter, the fetal trophoblast can be triggered to switch from an invasive to a proliferative phenotype using factors known to induce proliferative behavior in these cells. Proliferating cells divide and increase their number to a desired level, such as a number of cells sufficient to satisfy the sensitivity requirements of existing analytical technologies. These methods, involving inducing a switch from an invasive phenotype to a proliferative phenotype in order to selectively culture trophoblasts (or other cells) can be used for noninvasive prenatal diagnosis of any disorder which can be diagnosed using the cultured fetal cells.

In order to facilitate the phenotype switch described herein, the particle-separating device described herein is modified by adding, in one example, a coated or uncoated porous membrane between the body and the cover of the device. The device is operated as described herein to segregate cells, and by additionally performing steps to separate and amplification cells as described in this example, which exploit the ability of fetal cells to invade, migrate, and proliferate.

Fetal Cell Segregation and Enrichment, General

The process described elsewhere herein is modified as follows. The apparatus described herein is used to segregate and enrich fetal cells from a sample (e.g., a maternal blood sample) as described herein. The segregated cells are disposed in the void 11 between the body 10 and a membrane 81. The invasive phenotype of the fetal cells is induced (e.g., by adding to the void compounds known to induce such a phenotype), which causes the fetal cells move away from the void that contains contaminating maternal cells across the membrane. Migrating fetal cells move to the pores 82 of the membrane 81 and invade through membrane 81 into a space between the membrane 81 and the cover 12. Within this space, the phenotype of the fetal cells is again switched, this time to a proliferative phenotype, which causes the cells to divide and increase in number. The resulting expanded population of fetal cells can be used, for example, for fetal diagnostic methods that require fetal genetic material. Because these cells can be obtained from samples that can be obtained non-invasively from the fetus (e.g., from a maternal peripheral blood sample), these methods have an advantage relative to other fetal diagnostic methods in that no disturbance of the fetus is required in order to obtain a fetal sample.

The apparatus and methods described in this example are useful for cells and particles other than fetal cells as well. They can be used not only for fetal cell separation, but also for segregation of bacteria, virus, protozoa, multi-cellular organisms such as worms, insects, parasites, mineral and organic particles, organic and inorganic molecules.

The membrane or other barrier 81 can be porous or non-porous, and can be made of one or more materials and/or coated with one or more materials. The purpose of the membrane or other barrier 81 is to provide a structure that enables at least two types of cells in a population to be segregated based on the ability of at least one type of cells in the population to bind therewith and/or pass therethrough. Obviously, multiple membranes or other barriers 81 can be used (either sequentially or in parallel, from the vantage point of a particle in the void 11) in the methods and apparatus described herein.

Fetal Cell Segregation and Enrichment, Background

Fetal trophoblast are very rare in maternal peripheral blood. In order to be useful for most existing analytical technologies, fetal cells in a sample must be substantially segregated from non-fetal (e.g., maternal) cells. Currently available fetal cell collection methods and devices are often unable to produce samples having sufficient numbers and purity of fetal cells. Useful fetal cells need not be cells that existed in the corresponding fetus; for many applications, it is sufficient if the fetal cells are progeny of cells obtained from the fetus.

Normally, primary fetal cells (e.g., cytotrophoblasts in placenta) pass phylogenetically through at least two different phenotypes. At an early stage, fetal trophoblasts exhibit a proliferative phenotype in which they grow, divide, and multiply. Later, they differentiate into an invasive phenotype and move from fetal side to the maternal side of the placenta (i.e., they invade into or through the placenta). It is believed that fetal trophoblasts that exhibit the invasive, migratory phenotype are the ones that reach the maternal bloodstream, e.g., as they migrate up the inner (endothelial) surface of maternal spiral arteries.

This process of phenotype switching in fetal trophoblasts is triggered by a combination of conditions, including by changes in oxygen concentration, and by occurrence of signal molecules that cause, induce, or support the phenotypic changes. These conditions and signal molecules can be used to induce phenotype switching in fetal trophoblasts in vitro, such as in the apparatus and methods described herein.

Fetal Cell Segregation and Enrichment, In Vitro Expansion

The methods and apparatus described herein exploit the natural abilities of early fetal cells to proliferate and invade, for the purpose of enabling segregation and culture of fetal trophoblasts. This is achieved by first triggering fetal trophoblast cells in a sample to invade into and migrate through a porous membrane (e.g., using known methods such as those described in Logan et al., 1992, Cancer Res. 52:6001-6009 or Yang et al., 2009, J. Histochem. Cytochem. 57:605-612). Trophoblast invade through the pores leaving behind contaminating maternal nucleated cells which are incapable of such invasion. The trophoblasts are thereby segregated from those contaminating cells.

Segregated fetal trophoblasts are induced to switch from the invasive phenotype to a proliferative phenotype using factors known to induce proliferative behavior (e.g., one or more of those described in Red-Horse et al., 2004, J. Clin. Invest. 114:744-754; Ray et al., 2009, Placenta 30:96-100; Genbacev et al., 1997, Science 277:1669-1672; Gobble et al., 2009, Placenta 30:869-875; Truman et al., 1986, “The Effect of Substrate and Epidermal Growth Factor on Human Placental Trophoblast Cells in Culture, Springer, Berlin; Zhou et al., 2008, “Extreme Makeover: Converting One Cell into Another,” Elsevier, Cambridge; U.S. Pat. No. 7,244,707). Proliferating cells divide and increase in number to a selected level, such as a level sufficient to satisfy the sensitivity requirements of existing commercial analytical technologies.

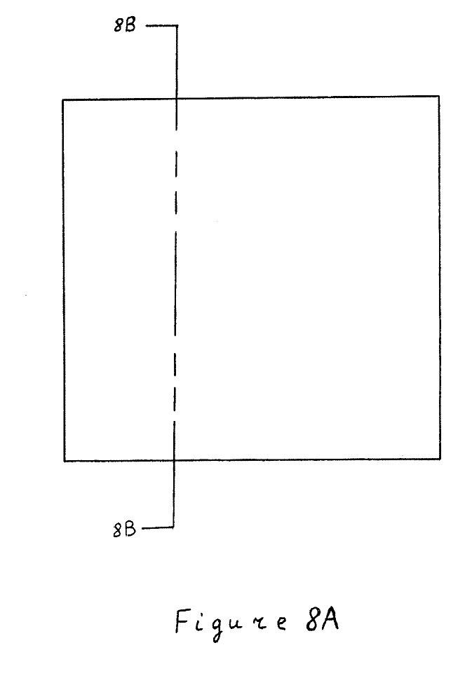

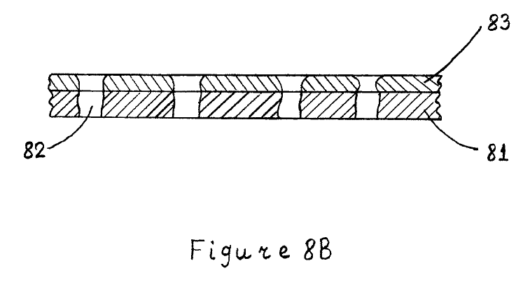

FIG. 8 illustrates a suitable embodiment of the membrane or other barrier 81. FIG. 8A is an elevated view of one embodiment of a portion of membrane 81. FIG. 8B is a magnified view of the portion of the vertical section of the membrane 81 from FIG. 8A, taken along plane 8B. In this particular embodiment, membrane 81 is porous and coated on one side, therefore FIG. 8B shows pores 82 extending through membrane 81 and coating 83. In embodiment shown in FIG. 8, the coating 83 is applied to one face of membrane 81, but not the other face, does not extend into pores 82, and does not fill pores 82.

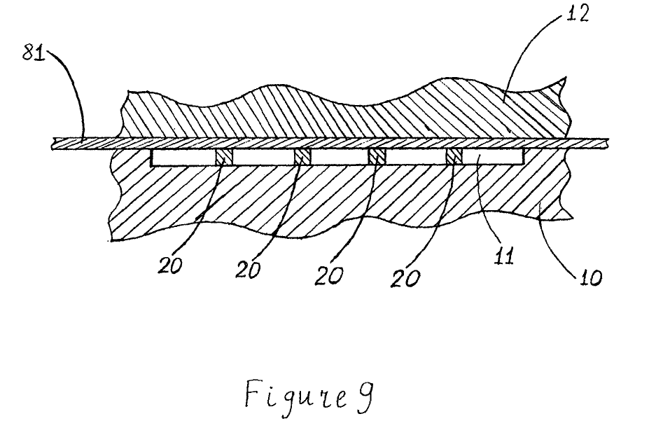

FIG. 9 illustrates a device incorporating a membrane or other barrier 81. FIG. 9 is a vertical section, taken along plane 9-9 in FIG. 2A, of a device of the type shown FIG. 2A and including a membrane 81 interposed between the body 10 and the cover 12. Inner supports 20 aid in even, leveled displacement of membrane 81 within the device. Inner supports 20 also help to define the void 11, and provide control over the size of the void 11. After cells are segregated, a population of cells, including fetal trophoblasts, remains within the void, in fluid communication with the membrane 81. A reagent disposed on the membrane (e.g., the B19 VP2 protein) causes fetal trophoblasts to bind with the membrane. Non-binding cells are removed from the void, for example by flushing it with a fluid that removes such cells. The void is then filled with a culture medium and with one or more agents capable of inducing the proliferative phenotype in the fetal trophoblasts. The trophoblasts proliferate on the membrane 81 and/or within the void 11 and can be harvested therefrom.

In another embodiment of the device partially illustrated in FIG. 9, instead of the cover 12 there is a second, identical body 10 with inner support structures 20. However, this second body is applied in the ‘mirrored’ fashion to the other side of the membrane 81, so the two identical congruent bodies “mirror” each other on both sides of the membrane. This creates a plurality of voids segregated from one another and contacting opposite faces of the membrane. In this embodiment the voids 11 on both sides of the membrane provide space for various species, such as cells, molecules etc., to permeate or invade through membrane from one void to the opposite void (i.e., across the membrane). Thus, if the membrane is made of a material (e.g., a thin film of basement membrane matrix) through which fetal trophoblasts can migrate in their invasive phenotype and a population of cells including invasive fetal trophoblasts occurs on one face of the membrane, penetration (i.e., invasion) of fetal trophoblasts can occur, resulting in appearance of fetal trophoblasts (but not other cells of the population) in the void on the opposite side of the membrane. This embodiment also provides the opportunity to introduce or withdraw non-identical fluids or samples from the voids on opposite sides of the membrane.

In vitro segregation and expansion of fetal trophoblasts capable of penetrating through a membrane 81 can be accomplished as follows, for example.

Fetal cells and other similarly-sized cells can be isolated from a maternal peripheral blood sample using the apparatus as described herein (ignoring disclosures relating to the membrane or other barrier 81). The size-segregated population of cells can be washed with fluid to substantially eliminate red blood cells, platelets, other small cells, and plasma. At this point, at least some of the cells in the population are fetal trophoblasts, but there may be maternal cells (e.g., large nucleated lymphocytes) in the population as well. Nonetheless, this population of cells, which can be retained within the void 11 of the apparatus, is significantly (e.g., 1000-fold) enriched for fetal cells, relative to the original maternal blood sample.

An agent is added to the void, the agent inducing fetal trophoblasts in the population to exhibit an invasive phenotype. By way of example, the void can be supplied with a gas mixture (i.e., in place of or above liquid in the void) containing up to 20% oxygen. This treatment renders the fetal trophoblasts capable of penetrating through a membrane 81 with which they are brought into contact (e.g., if the membrane 81 contacts the void 11, or if trophoblasts are recovered from the void 11 and contacted elsewhere with the membrane 81). If necessary or desired, the apparatus (or the portion of the apparatus containing the desired cells can be rotated or otherwise manipulated to encourage the cells to exhibit an invasive phenotype. Alternatively or in addition, an agent that induces migration of fetal trophoblasts toward the agent (e.g., the Smurf 2 gene product) can be sequestered on the side of the membrane 81 opposite the void 11 side of the membrane 81, thereby inducing fetal trophoblasts to migrate toward or through the membrane.

After the fetal trophoblasts have penetrated through the membrane into a second void or into a second material, they can be collected. Alternatively, if a greater number or concentration of fetal trophoblasts is desired, the second void or the second material (or, optionally, the entire apparatus) can be subjected to conditions (e.g., reduced oxygen concentration or addition of factors such as certain kinases known to induce a proliferative phenotype) that induce the trophoblasts to proliferate. Regardless of whether the cells are induced to proliferate prior to harvesting, the cells can be tested with a variety of methods and reagents (e.g., by analyzing their physical characteristics or their ability to bind with the B19 VP2 protein described herein) to confirm their identity as fetal trophoblasts.

The membrane 81 described herein can be made from, or coated with, a formulation resembling an extracellular matrix of human placenta, or from reconstituted basement membrane-like matrix. Commercially available products for making such membranes and coatings include Matrigel™ (Collaborative Research, Lexington, Mass.); and basement membrane extract Cultrex™ (Trevigne, Inc., Gaithersburg, Md.). As shown in FIG. 8B pores which can run continuously from one side of membrane to the other, thus fluidly connecting the opposite planar faces of the membrane to each other (and fluidly connecting the voids on either face of the membrane). Pore size and shape as well as their coating or filling can depend on particular application, device design and on type of particle, cell or molecule, etc., being separated.

Another embodiment of a suitable apparatus can be described by reference to FIG. 9. In this embodiment, instead of body 10 can include or carry an additional support-and-supply layer between the membrane 81 and cover 12. This layer can, for example, be made from rigid molded or extruded plastic or elastic silicone rubber. This layer provides two necessary functions for this integrated device. First, this layer can supply structure—to provide the set of supply channels for circulating culture media, and also provide room for expansion of proliferating/dividing target cells. Second, this layer can provide mechanical support such as ridges similar to the inner support structures 20 on FIG. 9. The membrane can be clamped between these ridges and the support structures 20. This way, transition of force will be provided from cover 12 through ridges to membrane 81 to the support structures 20 and to the body 10, to ensure flat uniform clamping and stretch of the membrane suspended between the cover and the body of the separation device.

The membrane 81 can include shaped portions to direct flow of fluid, migration of cells, or both. In one embodiment, a first layer 83 coating membrane 81 is sheer, and a second layer is deposited, printed (or scribed) on the first layer (i.e., on the face opposite the face of the first layer that contacts the membrane 81) to create elevated ridges and recessed troughs (channels). The width of strips and troughs can be selected based on how far the cell processes would be expected to reach, so that cells can reach and sense the troughs with their processes, then migrate into troughs if there are factors attracting them, such as flow of medium in the troughs. Alternatively, one or both of the body 10 and the cover 12 can have grooves or other shaped surfaces providing the same functionality. The membrane 81 can, of course, be shaped or cut to fit the void 11 between the body 10 and cover 12, and can have holes or fittings to accommodate other elements of the apparatus (e.g., inlet and outlet ports).

The membrane or other barrier 81 can have a uniform thickness, or the thickness can vary. One or both faces of the membrane or other barrier 81 can have a shape, such as grooved throughout or molded in multiple repeated patterns, depending on particular application, device design and on type of particle, cell or molecule being separated. Such multiple pattern variations could also be applied to changes in the material, porosity, and coatings of the membrane, that could correspond or not to the above mentioned structural and geometric variations.

Two membranes or other barriers 81 having a layer of a matrix between them can be used in place of a single membrane or other barrier 81. The edges of membranes or other barriers 81 can be sealed together, effectively creating a flat pouch. The matrix can contain, for example, culture media and/or compounds that induce an invasive or proliferative phenotype in fetal trophoblasts. By way of example, if the matrix includes a cell culture medium and a factor capable of inducing a proliferative phenotype in fetal trophoblasts, then invasive trophoblasts that enter the matrix can be converted to a proliferative phenotype and induced to proliferate in the cell culture medium.

In an alternative embodiment of methods of selectively segregating and proliferating cells, bacteria of a selected type (e.g., a human pathogen) can be segregated from a sample taken from a human and induced to proliferate in an apparatus described herein.

Bacteria are very small and are often difficult to separate in microfluidic devices. However, bacteria can be separated using an apparatus described herein that includes a cell separation apparatus that segregates cells based on their ability to pass through portions of the device and that also includes a porous membrane interposed between the void 11 of the apparatus and a medium that selectively attracts one or more bacteria of interest and/or promotes proliferation of the bacteria. If the membrane fluidly communicates with a fluid in a void 11 of the apparatus, then bacteria in the void can move (actively or passively) across the membrane and proliferate in the medium. Such apparatus and methods can be useful for detecting small numbers of bacteria in a sample (e.g., sparse populations of bacteria or other microbiological pathogens in samples such as blood, urine, food products, wastewater, etc.).

In addition to including attractants or culture media on the side of the membrane or other barrier 81 opposite the void 11, other apparatus or compositions can be located in that space. By way of example, that space can be used as a reaction/detection compartment, for example filled with gel, for gel electrophoresis of DNA. By way of example, bacteria that have migrated into the gel can be lysed and their nucleic acids can be electrophoretically separated or reacted or hybridized with other reagents.

Isolation of Fetal Cells from Maternal Arterial Blood Samples

Human fetal trophoblasts are believed to exhibit cell diameter generally in the range 14.3 to 30 micrometers. The lumen of mammalian capillaries can exhibit a significantly smaller diameter, on the order of 15 micrometers or smaller (see, e.g., Wang et al., 2007, Exp. Eye Res. 84:108-117, in which microspheres having a diameter>8 micrometers administered to arterial blood were observed not to reenter systemic circulation; Maxwell et al., 1985, Heart Circ. Physiol. 248(2):H217-H224 similarly observed a size limit of about 9 micrometers for arterially-administered microspheres passing through intestinal capillary circulation).

In view of these observations, it was determined that fetal trophoblasts (and similarly large cells) in vivo will be selectively concentrated on the arterial side of systemic capillary beds. It follows from this determination that fluids that are on the arterial side of blood capillaries are particularly suitable sources of fetal trophoblasts. Such fluids can include arterial blood, especially arterial blood taken upstream (with respect to physiological blood flow) of capillary beds. These fluids can also include fluids derived from arterial blood prior to passage of the arterial blood through capillaries (e.g., lung or bronchial secretions) or other narrow passages (e.g., blood in the common hepatic artery).

The observations described in this example also indicate that significant numbers of fetal trophoblasts (and similarly large cells) can be expected to accumulate in capillary beds, especially on the arterial side of such capillary beds. Blood taken immediately upstream from capillary beds can be expected to be relatively enriched in fetal trophoblasts (and similarly large cells).

In view of the relative rarity of fetal trophoblasts in maternal circulation, collecting fetal trophoblasts from bodily locations in which they are enriched can make the difference between detection and non-detection of such cells. Thus, the observations in this example suggest that obtaining samples of maternal arterial blood, fluids derived from arterial blood (i.e., prior to such blood passing through capillaries), or maternal blood collected immediately upstream of capillary beds can improve the likelihood that fetal trophoblasts (and similarly large cells) can be collected from such samples, relative to venous blood samples.

Use of a Cell Surface Marker for Fetal Trophoblasts

Others have reported that the glycosphingolipid designated globoside occurs on the surface of fetal trophoblasts and a limited number of other cells, and that globoside acts as the receptor for human parvovirus B19. The VP2 capsid protein of B19 specifically binds with cell-surface globoside. Wegner et al., 2004, Infect. Dis. Obstet. Gynecol. 12:69-78. Wegner et al. have reported that empty B19 capsids bind specifically with human villous trophoblast cells.

Others have reported that B19 does not bind globoside alone, but instead binds a complex of one or more glycosphingolipids and/or other molecules. Kaufmann et al., 2005, Virology 332:189-198. Regardless of the exact identity of the entity with which B19 and its capsid proteins bind, B19 and its capsid proteins bind human fetal trophoblasts, and appear to require occurrence of globoside in the membranes of those trophoblasts for such binding. Thus, B19, its capsids and capsid proteins, and other globoside-binding agents can be used to identify and segregate human fetal trophoblasts from other human cells (including from human cells occurring in blood of pregnant and previously-pregnant mothers).

Globoside is widely expressed in humans. Evidently, only a small proportion of the human population fails to express globoside on their erythroid cells. Expression of the globoside reportedly is highest in trophoblasts of human fetuses in the first trimester of development. Thus, globoside can be used as a marker to identify fetal trophoblasts, and the intensity of globoside expression by a cell can be used as an indicator of the stage of development at which the trophoblast separated from the fetus, with relatively early-stage trophoblasts generally expressing globoside at a higher level (or cell surface density) than later-stage trophoblasts.

Relative to fluorescent in situ hybridization methods that identify fetal trophoblasts by hybridization with chromosomal material (i.e., of which only one to several copies exist per cell), cell detection methods that involve detection of globoside can have significantly greater signal-to-noise ratios, in view of occurrence of multiple globoside molecules on the surface of cells. Moreover, correlation of globoside expression with early stage of fetal trophoblast development permits selection or segregation of cells based on the intensity of globoside expression.

Globoside can be used as a cell surface marker to identify fetal trophoblasts and other globoside-expressing cells. Other features (e.g., cell size and conformation) can be used to differentiate trophoblasts from other cells (e.g., erythroid cells, megakaryocytes, endothelial cells, and fetal cardiomyocytes) that express globoside.

Any reagent that specifically reacts with or binds to globoside can be used to identify globoside-expressing cells, including for example, a monoclonal antibody raised against globoside, human parvovirus B19 VP2 capsid protein (e.g., chromogen-, radio-, or biotin-labeled protein), empty B19 capsids, or intact B19 virus. Such reagents can also be used as capture reagents, for example, by adhering the reagent to a surface of the device described herein such that globoside-expressing cells will adhere specifically to that surface.

### EXAMPLES

The subject matter of this disclosure is now described with reference to the following Examples. These Examples are provided for the purpose of illustration only, and the subject matter is not limited to these Examples, but rather encompasses all variations which are evident as a result of the teaching provided herein.

### Example 1

### Separation of Fetal Cells from Maternal Blood

An apparatus of the type disclosed herein was used to separate fetal-like, large nucleated cells from other cells in a 1 milliliter sample of maternal blood.

The polycarbonate apparatus was constructed using a known epoxy resin casting process and included a body 10 having an integral separation element 14 in each of eight channels defined by the body 10. Other materials acceptable for this application include cyclic olefin copolymers, and polypropylene cyclo-olefin polymer.

The separation element had six steps defining serially-arranged passages in a stepped passageway, the passages having narrow dimensions of 10, 7, 5, 4, 3, and 2 micrometers, respectively. Each step (and passage) had a length of 1 millimeter. A standard glass microscope slide clamped to the body 10 was used as a cover 12. Portions of the body 10 between the discrete stepped passageways served as supports 20. The cover 12 was bonded to the body 10 using silicone rubber adhesive.

In order to simulate maternal blood, a sample of blood from a male fetus was obtained and mixed with blood obtained from a woman. This mixture was heparinized using a standard procedure and refrigerated overnight. Other anticoagulants, such as potassium EDTA, are also suitable for this application. The sample was brought to room temperature and injected into the inlet region 15 of a plurality of channels using a syringe. After the sample passed through the apparatus, the apparatus was observed under a microscope. Large cells (i.e., cells larger than normal blood cells) that appeared to be of fetal origin were observed to have been trapped as several positions within the stepped passageways.

The large cells were adhered to the glass cover by briefly centrifuging the assembled apparatus. Following centrifugation, the cover 12 was removed from the body 10 and cells adhered to the cover 12 were fixed by Carnoy fixation using a 3:1 mixture of methanol:acetic acid. The cells were then processed with a standard fluorescence in-situ hybridization (FISH) protocol for detection of chromosomes X and Y using a commercially available kit.

Fluorescent signals representing the hybridization of the FISH probe to site specific sequences on the X and Y chromosomes were observed on the slide, indicating that male (i.e., Y chromosome-containing) fetal cells had been segregated from the blood sample using the apparatus. At least some of the large cells were observed to be polynucleate, suggesting a trophoblastic origin.

Fetal trophoblastic cells are believed to be eliminated from maternal blood relatively rapidly following cessation of pregnancy, unlike other types of fetal cells that may occur in maternal blood (e.g., primitive fetal stem cells). Because trophoblastic cells from previous pregnancies are unlikely to persist in the blood of women, segregation of fetal trophoblastic cells can be more informative regarding the status of the woman's current fetus than segregation of other types of fetal cells (including those which may have persisted from previous pregnancies, known or unknown to the woman).

### Example 2

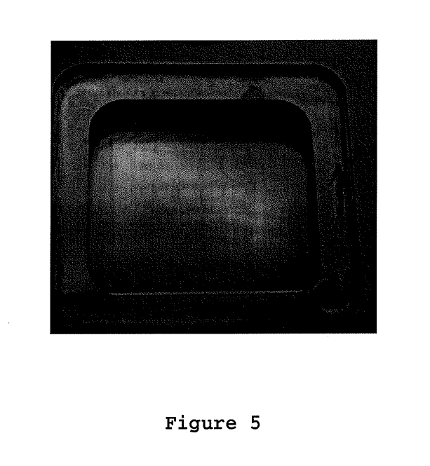

Assessing assembly of an apparatus described herein can be achieved by observing light reflected, refracted, or both reflected and refracted from the apparatus under illumination. FIG. 5 is a color image which depicts the pattern of light observed on an appropriately assembled apparatus.

The apparatus shown in FIG. 5 is formed of a plastic body having a separation element integral therewith and having a flat glass cover applied thereto. A stepped passageway is defined by the cover on the (here) upper face of the stepped passageway and by the separation element on the (here) lower face of the stepped passageway. Nine supports extend substantially the entire length of the separation element, from the inlet region (in the direction of the arrow shown in FIG. 5) to the outlet region, dividing the stepped passageway into 10 separated flow channels. The separation element has eight flat portions essentially parallel to the cover, the flat portions (steps) defining distances of 4.0, 4.2, 4.4, 4.6, 4.8, 5.0, 5.2, and 5.4 micrometers from the surface of the cover that defines the stepped passageway.

Fluorescent light was emitted from a source at an angle of illumination approximately perpendicular to and directly above the cover. FIG. 5 shows the image observed by an observer positioned with a line of sight at approximately 30-45 degrees to the cover. It can be seen that a “checkerboard”-like pattern of light is observed, as shown in FIG. 5. Without being bound by any particular theory of operation, it is believed that light reflected from the top (i.e., outside the stepped passageway) surface of the cover combines with light reflected by the bottom surface of the cover, light reflected by the flat portions of the separation element, or some combination of these to yield the colors seen in FIG. 5. Regardless of the origin or explanation of the light variations, a pattern of light corresponding to the pattern of the separation element and supports is observed when the apparatus is assembled appropriately. Deformations in the cover or body, for example, distort the checkerboard pattern, such that the rectangles corresponding to flat portions of the separation element appear lopsided or curved.

### Example 3

### Isolation of Fetal Cells from a Human Chorionic Villus Sample

In the experiments described in this example, an apparatus of the type described in this application was used to segregate fetal cells from a mixture of adult and fetal cells that was present in a chorionic villus (CV) sample obtained from a pregnant woman known to be carrying a male fetus.

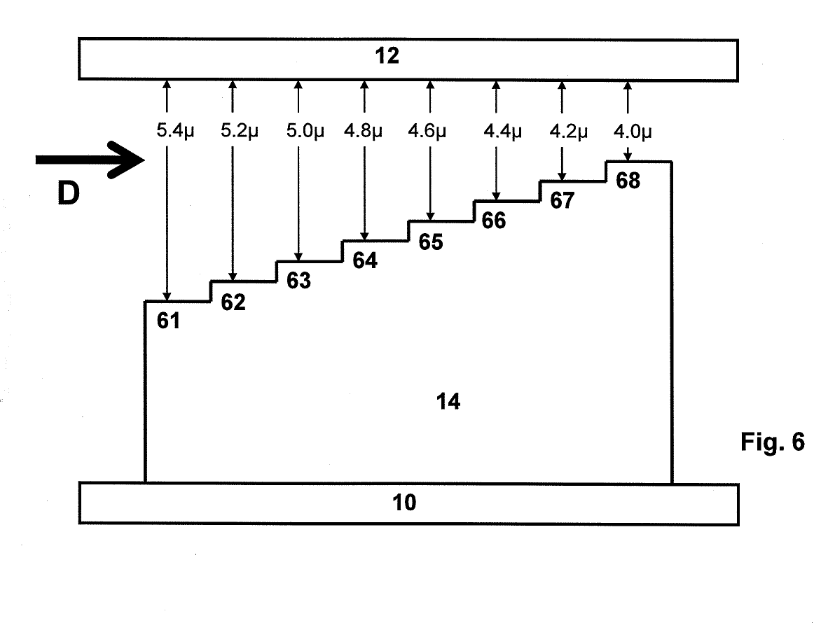

The apparatus used in the experiments described in this example was a two-piece cassette having a body manufactured from polycarbonate using a micro-injection molding process and a glass cover, the body having a separation thereon defining multiple steps between the separation element and the cover, as shown in FIG. 6. The body and cover of the cassette defined a void having an inlet region and an outlet region. The inlet and outlet regions were in fluid communication with each other by way of a separation region. The separation region included a flat segment (i.e., a relatively broad passageway) wherein the minimum distance between the body and the cover was 4.0 micrometers and the maximum distance between the body and the cover was 5.4 micrometers. The cover-to-step distances for the eight steps were (in the direction of fluid flow) 5.4 micrometers, 5.2 micrometers, 5.0 micrometers, 4.8 micrometers, 4.6 micrometers, 4.4 micrometers, 4.2 micrometers, and 4.0 micrometers, as shown in FIG. 6. The length of the separation region in the direction of fluid flow (i.e., the distance, left-to-right of the 8-stepped structure shown in FIG. 6) was 20 millimeters, and each of the eight steps within the separation region had a length, in the direction of fluid flow, of 2.5 millimeters. The width of the separation region (i.e., the distance that the 8-stepped structure shown in FIG. 6 extended in the dimension perpendicular to the planar view shown in FIG. 6) was 24 millimeters. The total internal volume of the void of the assembled apparatus was about 12.2 microliters, with the volume of the separation region of the void (i.e., the portion between the cover and the stepped separation element) being about 2.2 microliters and the combined volumes of the inlet and outlet regions being about 10 microliters. This model of cassette was designated D3v2.

In an alternative embodiment, a similar apparatus can be used, the apparatus differing substantially only in that the cover-to-step distances for the eight steps are (in the direction of fluid flow) 4.4 micrometers, 4.2 micrometers, 4.0 micrometers, 3.8 micrometers, 3.6 micrometers, 3.4 micrometers, 3.2 micrometers, and 3.0 micrometers. This model of cassette is designated D2V3.

During fluid flow operations, the cassette was contained within a purpose-designed holder that served to clamp the cassette and ensure that the glass cover mated with the cassette body in a manner that prevented leakage of any fluid from the cassette. The precise construction of the holder was not critical, and served to apply pressure evenly to the parts of the cassette sufficiently to hold them together and prevent leaks due either to positive or negative fluid pressure within the cassette, relative to atmospheric pressure. For the experiments described in this Example, the holder was constructed of two metal parts having fittings for adjusting the force with which the metal parts and the cassette parts sandwiched between them were held together. One of the metal parts defined a ‘window’ (see FIG. 5) that approximately corresponded to the void region between the body and cover, through which visual observations of cells within the void could be made. The other metal part was substantially solid, except that it included holes aligned with the inlet and outlet ports to accommodate connections for providing fluid to and withdrawing fluid from the void within the cassette.

Fluid flow through the cassette was achieved using a Hamilton PSD3 syringe pump equipped with a 1.25-milliliter syringe. The pump was software-controlled using an application running on MatLab™ Instrument Control Toolbox. The system also includes a pressure sensor that enabled the fluid pressure within the cassette to be constantly monitored. The fluid conduits and fittings with which the components of the system were connected were selected to accommodate anticipated pressures, but their identity was not critical. Substantially any fluid conduits and fittings can be used.

The molecular probes used in these studies were obtained from Abbott Molecular and consisted of CEP® X Spectrum Orange™ probe (providing a red fluorescence signal from the X-chromosomes in cells treated with the reagent) and CEP® Y Spectrum Green™ (providing a green fluorescence signal from the Y chromosome occurring in cells treated with the reagent). All other reagents were of sufficient grade to prevent non-specific hybridization.

A CV sample was received in a 15-milliliter screw-cap plastic tube containing pieces of tissue in approximately 5 milliliters of Dulbecco's modified phosphate-buffered saline (DMPBS; 0.90 millimolar CaCl₂; 0.49 millimolar MgCl₂, 2.7 millimolar KCl, 1.47 millimolar KH₂PO₄, 138 millimolar NaCl, and 8.06 millimolar Na₂HPO₄ at pH 7.2) in which cells from the tissue sample were suspended. The cell suspension was aspirated, leaving the solid tissue fragments at the bottom of the tube (the volume of material remaining in the tube was less than 0.25 milliliter). The aspirate was placed in a 15-milliliter screw-cap plastic tube and centrifuged at 3,000 rpm (ca. 1,500×g) for 5 minutes. After centrifugation and removal of the supernatant, approximately 0.1 milliliter of packed cells remained in the tube. Approximately 2 milliliters of DMPBS was added to the tube, and the components were mixed using a vortex-type mixer sufficiently to re-suspend the pelleted cells. This re-suspended cell sample was stored at 4 degrees Celsius for approximately 1 hour.

A sample of the re-suspended cell sample was spread on a standard glass microscope slide, stained with Wright-Giemsa stain, and examined at a magnification of 400× under illumination with white light. The stained preparation showed the presence of (fetal) trophoblastic cells in the sample. Other cells that were observed in the sample were believed to be neutrophils (nucleated white blood cells) and red blood cells. Observations of fetal trophoblastic cells and other cells in the sample by this method revealed that the trophoblastic cells were significantly larger than most other cells in the sample.

A 1.25-milliliter aliquot of suspended cells and passaged through the D3v2 cassette by application to the inlet region, using the syringe pump apparatus described above. Prior to application of the sample, the cassette had previously been primed by passage of a quantity of DMPBS. This sample was passaged at a fluid flow rate of 0.025 milliliter per minute through the cassette. During sample passage, the pressure in the fluidics system was monitored and observed to vary within the range 4.6-6.8 psig.

After the sample had been passaged through the cassette, three 0.1-milliliter aliquots of a fixative solution (methanol:acetic acid in a 3:1 ratio) were passed through the cassette. A 10-minute period was permitted to elapse between passages of fixative solution. The cassette and holder were chilled by application of water ice to the apparatus during the processes described in this paragraph. Preceding steps were performed at room temperature (roughly 20 degrees Celsius).

Following fixation, the cassette was dried by applying a vacuum to the outlet region, which effected removal of all fluid from the void in the cassette. The cassette was stored overnight at 4 degrees Celsius. Following storage, the cassette was microscopically observed at 100× magnification. Several nucleated cells having a diameter greater than about 20 micrometers were observed in the separation region, some within the inlet region, and others at the first separation step in the separation region of the cassette. No cells having a diameter greater than about 20 μm were observed downstream from the first separation step in the separation region of the cassette.

The cassette was disassembled and the glass cover was removed and processed using a standard FISH protocol. The cover was examined using a fluorescence microscope equipped with a computer-controlled stage coupled with an automated detection algorithm. The cover was also stained with DAPI to enable visualization of intact nuclei (i.e., to confirm capture of cells). FISH and DAPI staining were performed as provided in the commercial kit obtained from Abbott Molecular (Chicago, Ill.).

Examination the DAPI- and FISH-stained cover indicated an abundance of nucleated cells on the glass cover. Most of the cells were observed in a relatively small area at the portion of the cassette corresponding to the steps having cover-to-step distances of 4.2 and 4.4 micrometers. These cells appeared to be stretched or otherwise deformed. Male cells (i.e., cells generating fluorescent signals corresponding to the presence of both an X chromosome and a Y chromosome) were present. We concluded that these cells originated from the male fetus of the pregnant woman. Female cells (i.e., cells generating fluorescent signals corresponding to the presence of an X chromosome, but lacking any fluorescent signal corresponding to the presence of a Y chromosome) were also detected. We concluded that these cells originated from the pregnant woman, rather than from her male fetus. Of the cells detected, none exhibited a multi-lobed nucleus, from which we concluded that the cells that were captured were not white blood cells.

### Example 4

### Isolation of Fetal Cells from Maternal Blood Samples

In the experiments described in this example, an apparatus of the type described in this application was used to segregate fetal cells from a blood sample obtained from the circulation of a pregnant woman known to be carrying a male fetus.

The apparatus used in the experiments described in this example was the D3v2 cassette described in Example 3, operated as described in that example. The molecular probes and staining procedures that were used were the same ones described in Example 3.

Blood was collected in pairs of approximately 5-milliliter aliquots by venous puncture from each of 22 pregnant women known (by ultrasound imaging) to be carrying a male fetus. The gestational age of the fetuses was within the range from 17 weeks, 6 days and 29 weeks, 6 days, with the average gestational age being 21 weeks, 5 days and the median age being 20 weeks, 2 days. Each blood sample was collected in a 5-milliliter tube and was stored in an ice bath until it was prepared for application to the cassette. The time that elapsed between sample collection and sample preparation was less than one hour.

In some instances, the same sample was passaged through two cassettes, one of which was subsequently stained using FISH reagents, and the other of which was stained using Wright-Giemsa reagents. This permitted comparison of histology (Wright-Giemsa stained cells) results and results obtained via FISH procedures. In other instances, duplicate blood samples from a single patient were passaged through separate cassettes, in order to confirm reproducibility of results.

Blood samples were passaged through the cassette by aspirating patient blood sample into the Hamilton syringe in preparation for pumping through the cassette. The 1.25-milliliter blood samples were pumped through individual cassettes at a flow rate of 0.025 milliliter per minute. The pressure in the fluidics system was monitored during blood sample passage, and was observed to vary within the range 7-9 psig.

Following passage of the blood sample through a cassette, 1.25 ml of Dulbecco's modified phosphate buffered saline was passaged through the cassette in the same direction and at the same flow rate as the sample flow in order to remove any residual material from the sample, other than cells retained in the cassette. After this wash procedure, three 0.1-milliliter aliquots of the fixative was passaged through the cassette at 0.025 milliliter per minute. A 10 minute period was permitted to elapse between the fixative passages. The cassette and holder were chilled by application of water ice to the apparatus during passage of the fixative aliquots and the intervening periods.

Following fixative passages, cassettes were treated in one of two ways. Some cassettes had the fixative removed immediately after passage (by passage of filtered air through the cassette until the cassette was free of fixative droplets), were stored overnight at 4 degrees Celsius, and FISH-treated after overnight storage. Other cassettes were stored at 4 degrees Celsius with the fixative retained within the cassette until four or more cassettes had been accumulated, at which time the fixative was removed, the cassettes were stored overnight at 4 degrees Celsius, and the cassettes were FISH-treated following the overnight storage. FISH-treatment entailed removal of the cover and processing using the CEP® X Spectrum Orange™ CEP® Y Spectrum Green™ as described in Example 3. DAPI was used as a counter-stain and to demonstrate the presence of an intact nucleus.

After staining, the glass cover having the stained cells attached thereto was examined using a fluorescent microscope either manually or using a computer-controlled stage coupled with an automated detection algorithm.

For some cassettes, cells fixed onto the cover were stained only with Wright-Giemsa stain in order to examine the types and distribution of cells captured within the cassette.

The results obtained from the experiments in this example are now discussed.

Samples of maternal blood obtained from 22 pregnant women was passaged through 38 cassettes. Twenty-six (26) cassettes were processed using the FISH/DAPI procedures described herein and 12 cassettes were stained only with Wright-Giemsa. Of the 26 cassettes used for FISH, 12 were found to be suitable for analysis and 14 failed to hybridize correctly or to pick up the counterstaining, indicating that they were improperly fixed.

Of the 12 cassettes that were suitable for analysis, 3 provided a male-positive signal when approximately 12.5% of the total cover area was scanned using the automated microscope and algorithm. Due to concerns about the rigor of the automated system, some cassettes were re-scanned manually. Re-scanning of the cassette revealed occurrence of male-positive-signal cells on each of the 12 cassettes, with between one and eleven male cells detected on the individual plates (the numbers of male cells detected on each of the twelve plates were 1, 2, 2, 2, 2, 3, 3, 3, 3, 4, 8, and 11). Of those male cells, most (64%) were detected on the portion of the cassette cover corresponding to the steps having cover-to-step distances of 4.0, 4.2, and 4.4 micrometers. Approximately 36% of male cells were detected on the portion of the cassette cover corresponding to the steps having cover-to-step distances of 4.6, 4.8, 5.0, 5.2, and 5.4 micrometers.

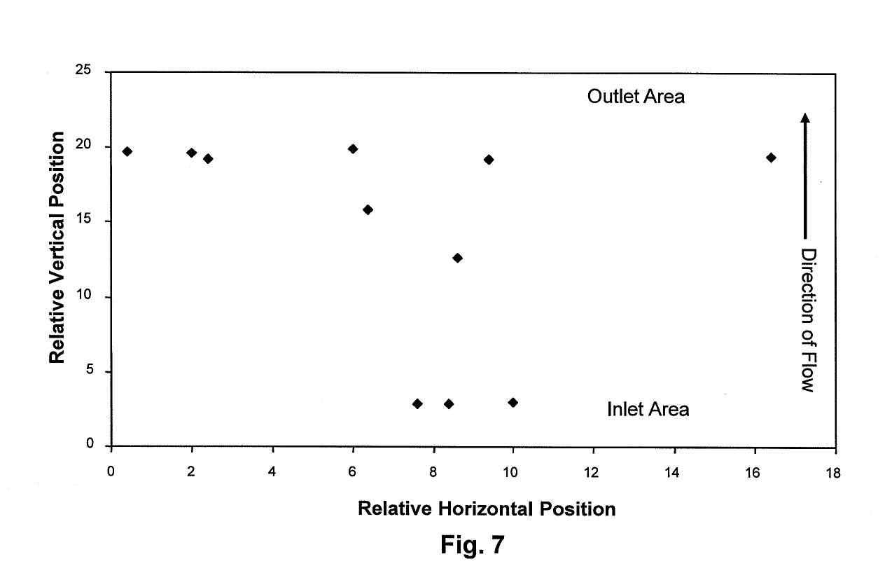

FIG. 7 provides a relative “map” of the location of each of the identified cells that provide a positive signal for a male fetal cell. Most of the identified cells are at the exit or outlet portion of the cassette with a few of the cells in the inlet area. This indicates that the cassette is capable of capturing fetal cells and does not permit their passage.

Results from one cassette indicated that 11 fetal cells (i.e., cells exhibiting fluorescent signals indicative of the presence of both X and Y chromosomes in their nuclei) were captured, as were fewer than about 300 adult female cells (believed to be primarily white blood cells).

Twelve cassettes were stained with Wright-Giemsa stain to examine the morphology of captured cells. These cassettes were not used for FISH analysis and were observed only by light microscopy. Two of these cassettes were provided to an expert in nucleated white blood cells (a transplantation immunologist) who was not informed as to the nature of the sample that had been applied to the cassettes. This expert opined that the captured cells included an irregular band of predominately “epithelioid cells” having granulocytes and mononuclear cells intermingled therewith. Although the cytological morphology of these cells was described by the expert as epithelial-like, they were believed to be trophoblasts or other large cells, in view of the fact that the immunologist was not told to expect that fetal trophoblasts might be among the cells observed. Fetal trophoblasts are known to be epithelial cells that invade maternal blood vessels in the placenta.

Fetal trophoblast-like cells were observed at the portion of the cassette cover corresponding to the steps having cover-to-step distances of 4.0, 4.2, and 4.4 micrometers, where the majority of the cells that provided a signal for both X and Y chromosomes were found. The estimated frequency of these trophoblast-like cells was much higher than would be expected for circulating cancer cells or for other cells of similar morphology in normal blood. This observation indicates that the cassette captures cells that are not normally observed (or are observed only in very low numbers) in human circulation.

Examination of the cassettes used for the experiments described in this example indicates that the cassette typically captured between about 200 to 4,000 cells from each 1.25-milliliter sample of maternal blood. It is apparent from observations of the captured cells that at least some of the captured cells were cells of fetal origin. However, it is equally apparent that the cassettes are able to capture a variety of other blood-borne cells from blood samples. These other cells include white blood cells. Analysis of the positions at which cells were captured in the cassettes used in these experiments revealed that cells were captured primarily at three distinct regions. Approximately 30-35% of cells were captured at the portion of the cassette at which the steps having cover-to-step distances of 5.2 and 5.4 micrometers occurred. Approximately 25% of cells were captured at the portion of the cassette at which the steps having cover-to-step distances of 4.0, 4.2, and 4.4 micrometers occurred. The remainder of captured cells were captured at portions of the cassette corresponding to the intervening steps (i.e., those having cover-to-step distances of 5.0, 4.8, and 4.6 micrometers)

Under the conditions used in these experiments, it was observed that captured neutrophils (which generally have a cell diameter of about 9-10 micrometers) were able to migrate further along the separation chamber of the cassette in the direction of fluid flow than were monocytes (which generally have a cell diameter of about 10-30 microns), which were more frequently retained nearer the upstream side of the separation chamber. These observations indicate that the apparatuses described in this application can be used both to segregate fetal cells from maternal blood cells and to segregate different types of maternal blood cells. The observation that (relatively larger) monocytes tend to be more frequently captured nearer the upstream portion of the separation chamber than (relatively smaller) neutrophils supports the contention that the ability of cells to traverse the separation chamber is inversely size-dependent. Thus, these observations indicate that the results can be extrapolated beyond blood-borne cells to predict that cells, whether they be blood cells or not, (and, particles other than cells) can be segregated by size using apparatus such as those described herein.

Another interesting observation that was made in the experiments described in this example relates to preferential retention within the cassette of monocytes over neutrophils, relative to their relative frequencies of occurrence in blood. The populations of neutrophils and monocytes within a normal blood sample are generally in the range 50-70% and 2-8%, respectively. That is, in normal blood, neutrophils tend to outnumber monocytes by an order of magnitude or more. However, in the experiments described in this example, the ratio of neutrophils:monocytes was more nearly (55-65):(35-45). The ratio of neutrophils:monocytes is far higher (1.03:1 on the upstream side of the separation chamber and 1.67:1 on the downstream side) in samples obtained using the devices described herein than the ratio that occurs in normal blood (approximately 50:1). These results indicate that the apparatus described in this application, at least when configured as described in this example, capture monocytes more effectively than they capture neutrophils.

Assuming that there are between 4 and 10 million white blood cells within a milliliter of maternal blood, the experiments described in this example demonstrate that the use of the cassette described herein eliminated substantially all red blood cells, platelets and plasma, and more than 99% of all nucleated white blood cells, from a 1.25 ml sample of maternal blood, while retaining apparently segregable pools of several cells of potential interest, including fetal cells. If it is assumed that there are about 2.5-6.25×10¹⁶ cells in a 1.25-milliliter sample of blood, then the results discussed in this example demonstrate that the operation of the apparatus described herein resulted in passage through the cassette of essentially all of the cells, since the cassette capture only 553±316 (mean±standard error of the mean for an N of 6), while still retaining cells of interest. This degree of specific cell separation is remarkable—a roughly 10¹⁴-fold purification, even ignoring segregation of particles within the separation region.

The results of the experiments described in this example demonstrate that the cassettes described in this example are able to accommodate passage of blood through a narrow space, defined in one dimension in microns. In the devices described in this example, the fluid pressure and other characteristics which can disrupt cellular integrity and potential clog narrow passages due to “packing” of cells did not cause these effects on the blood samples that were used. Clotting of blood was also not observed. Without being bound by any particular theory of operation, it is believed that the cassettes described in this example provide appropriate distancing within the cassette to maintain a space sufficient to permit passage of all red blood cells, platelets and most white blood cells while providing a separation selection process dependent on size or diameter of the particles.

The studies described in this example defined flow conditions sufficient to passage 1.25 milliliters of blood through the cassette with minimal damage to cells and no clotting. Furthermore, passage of the blood sample was achieved in less than an hour, using only a single separation unit. This cell-separation time is substantially shorter than is achievable using other cell-separation methods and is sufficient to deliver defined sub-populations of blood cells within clinically- and commercially-relevant time periods. These rapid methods and the apparatuses used to perform them permit collection of cell populations of diagnostic interest, either for prenatal fetal diagnosis or other diagnostic, therapeutic or research applications. The ability to capture a whole fetal cell, while also allowing the vast majority of other cells to pass through the cassette, can provide a complete fetal genetic sample for analysis and detection of genetic abnormalities, for example. These data also demonstrate that material captured by the device is suitable for use in molecular diagnostic protocols.

The cassette and methods described in this example provide a valuable tool for the selection of cells and other particles of biological interest for therapeutics, diagnostics and general research applications where it is important to either enrich a cell or particle sample for analysis or obtain a pure population for analysis. Applications in genetics, phenotypic analysis epigenetic analysis are areas that could benefit from such isolation processes.

The disclosure of every patent, patent application, and publication cited herein is hereby incorporated herein by reference in its entirety.

While the subject matter has been disclosed herein with reference to specific embodiments, it is apparent that other embodiments and variations of this subject matter can be devised by others skilled in the art without departing from the true spirit and scope of the subject matter. The appended claims include all such embodiments and equivalent variations.

1. A method of segregating fetal-like cells from a maternal blood sample, the method comprising
introducing the sample at the inlet region of an apparatus comprising a body, a cover, and a separation element,
the body and cover defining a void that contains the separation element, the void having a inlet region and a outlet region, and
the separation element having a first step and a second step and defining a stepped passageway that fluidly connects the inlet and outlet regions,
the stepped passageway including a first passage bounded by the first step and at least one of the body and the cover and further including a second passage bounded by the second step and at least one of the body and the cover,
the first passage having a narrow dimension and being fluidly connecting the second passage and the inlet region;
the second passage having a narrow dimension narrower than the narrow dimension of the first passage, the narrow dimension of the second passage being in the range from 8 to 15 micrometers;
and inducing fluid flow in the stepped passageway in the direction from the inlet region toward the outlet region, whereby maternal blood cells traverse the first and second passages and pass to the outlet region and fetal-like cells are unable to enter the second passage.

2-20. (canceled)

21. An apparatus for segregating fetal-like cells from a sample,
the apparatus comprising a body, a cover, and a separation element,
the body and cover defining a void that contains the separation element, the void having an inlet region and an outlet region, and
the separation element having a first step and a second step and defining a stepped passageway that fluidly connects the inlet and outlet regions,
the stepped passageway including a first passage bounded by the first step and at least one of the body and the cover and further including a second passage bounded by the second step and at least one of the body and the cover,
the first passage having a narrow dimension and being fluidly connecting the second passage and the inlet region;
the second passage having a narrow dimension narrower than the narrow dimension of the first passage;
whereby fetal-like cells passing from the inlet region to the outlet region can be segregated by their inability to traverse either or both of the first passage and the second passage.

22. The apparatus of claim 21, wherein the narrow dimensions of the first and second passages are selected such that fetal-like cells in a fluid passing from the inlet region to the outlet region can be segregated by their ability to traverse the first passage and their inability to traverse the second passage.

23-24. (canceled)

25. The apparatus of claim 21, wherein the separation element is integral with at least one of the body and the cover.

26-29. (canceled)

30. The apparatus of claim 21, wherein the separation element defines a plurality of stepped passageways, each of which i) fluidly connects the inlet and outlet regions and ii) includes a first passage bounded by a first step and a second passage bounded by a second step.

31-34. (canceled)

35. The apparatus of claim 21, wherein the geometric dimensions of the first and second passages are selected to substantially equalize the linear flow velocities in the first and second passages of a fluid flowing through the stepped passageway.

36-42. (canceled)

43. The apparatus of claim 21, wherein the flow area of each of the first and second passages is rectangular.

44. (canceled)

45. The apparatus of claim 21, wherein the separation element defines at least three steps that bound sequentially narrower passages in the stepped passageway, in the direction from the inlet region to the outlet region.

46. The apparatus of claim 21, wherein the first passage is bounded by a planar surface of the separation element that is substantially parallel to a planar surface of one of the body and the cover.

47. The apparatus of claim 46, wherein the length of the planar surface of the separation element, in the direction of bulk fluid flow is at least four times the narrow dimension of the first passage.

48. The apparatus of claim 46, wherein the width of the planar surface, in the direction perpendicular to bulk fluid flow is at least 1,000 times the narrow dimension of the first passage.

49. A method of segregating particles, the method comprising
introducing particles suspended in a fluid at the inlet region of an apparatus comprising a body, a cover, and a separation element,
the body and cover defining a void that contains the separation element, the void having a inlet region and a outlet region, and
the separation element having a first step and a second step and defining a stepped passageway that fluidly connects the inlet and outlet regions,
the stepped passageway including a first passage bounded by the first step and at least one of the body and the cover and further including a second passage bounded by the second step and at least one of the body and the cover,
the first passage having a narrow dimension and being fluidly connecting the second passage and the inlet region;
the second passage having a narrow dimension and narrower than the narrow dimension of the first passage;
and collecting particles at the outlet region, whereby the particles at the outlet region have been segregated from other particles that are unable to enter the second passage.

50-69. (canceled)

70. The apparatus of claim 21, wherein the apparatus further comprises a barrier in fluid communication with the void, wherein the barrier comprises a material with which fetal-like cells interact differently than other cells in the sample.

71-73. (canceled)

74. The apparatus of claim 70, wherein the barrier is a membrane comprising a component of extracellular matrix of human placenta.

75. The apparatus of claim 70, wherein the apparatus further comprises a compartment bounded by the barrier and on the opposite face of the barrier from the void, whereby cells that pass through the barrier enter the compartment.

76. The apparatus of claim 75, wherein the compartment contains an agent that attracts fetal trophoblasts.

77. The apparatus of claim 76, wherein the agent is the SMURF2 gene product.

78-82. (canceled)

83. The apparatus of claim 1, wherein a portion of the apparatus defining the void is coated with a reagent that specifically binds fetal cells.

84. The apparatus of claim 83, wherein the reagent comprises VP2 protein of human parvovirus B19.

85-92. (canceled)

93. The apparatus of claim 21, wherein the narrow dimension of the second passage is in the range from 8 to 15 micrometers.
